(19) United States
(12) Patent Application Publication
(10) Pub. No.: US 2025/0352244 A1
(43) Pub. Date: Nov. 20, 2025

(54) TRANSVAGINAL ACCESS APPARATUS AND METHODS

(71) Applicant: MOMENTIS SURGICAL LTD., Or Yehuda (IL)

(72) Inventors: Shiran GOLAN, Tel Aviv (IL); Refael AVITAN, Ness Ziona (IL); Idan ROTEM, Tel Aviv (IL)

(21) Appl. No.: 18/871,570

(22) PCT Filed: Jun. 30, 2023
(86) PCT No.: PCT/IB2023/056805

§ 371 Date: Dec. 4, 2024

Related U.S. Application Data
(60) Provisional application No. 63/357,021, filed on Jun. 30, 2022.
(60) Provisional application No. 63/428,746, filed on Nov. 30, 2022.

(51) Int. Cl. A61B 17/42; A61B 17/34; A61M 13/00; A61M 29/02

(52) U.S. Cl. CPC A61B 17/42; A61B 17/3496; A61M 13/003; A61M 29/02

(57) ABSTRACT

A device for accessing a body cavity through a natural orifice comprises a proximal base member and a plurality of conduits arranged to receive a corresponding plurality of surgical tools. The device further comprises, for each conduit, a lumen shaped for passage therethrough of a surgical tool, the lumen having a proximal port and a distal opening, a gas port in fluid communication with the lumen and displaced distally from the proximal port, and a proximal sealing gasket seated in the lumen and adapted for selectively opening and closing. The gasket is displaced distally from the proximal port and proximally from the gas port so as to create a respective proximally-sealed distal gas volume in communication with the body cavity.

### CROSS-REFERENCE TO RELATED APPLICATIONS

This patent application claims priority from U.S. Provisional Patent Application No. 63/357,021 filed on Jun. 30, 2022, and from U.S. Provisional Patent Application No. 63/428,746 filed on Nov. 30, 2022, both of which are incorporated herein by reference in their entirety.

### FIELD OF THE INVENTION

The present invention relates to devices and methods for performing minimally invasive surgeries, and in particular to enabling transvaginal access for multiple surgical tools to surgical sites.

### BACKGROUND

It is well established that there are benefits of minimally invasive surgery. Instruments for such surgery typically have a surgical end effector located at the distal end of an articulated surgical arm (preferably with minimum diameter) that is inserted through a small opening (e.g., body wall incision, natural orifice) to reach a surgical site. In some instances, surgical instruments are passed through a cannula and an endoscope can be used to provide images of the surgical site. In some instances, a trocar needle is used to make an incision, and the incision is dilated, to enable access by surgical end effectors to a surgical site in the body cavity.

Available equipment for supporting minimally invasive surgeries can comprise numerous and disparate elements, and methods for using them are often complicated and not streamlined for efficiency. The state of the art of the available equipment means, for example, that replacement of a surgical tool (or of a surgical arm) during an operation requires the surgeon to remove all tools and arms from the body cavity before introducing or reintroducing them, e.g., in order to maintain sterility and status of insufflated gas. This drawback is exacerbated by gas systems for insufflation and expulsion not being associated with individual cannular conduits. As another example, the design of available cannulas does not take into account other instruments that need to access the body cavity besides the robotic surgical arms. As another example, needles and dilators generally need to be used and then removed completely from the body cavity to make way for other equipment items and/or the surgical arms. There is a need for improved designs of access devices, e.g., transvaginal access devices, for improving and streamlining the processes.

### SUMMARY OF THE INVENTION

According to embodiments disclosed herein, a device for accessing a body cavity through a natural orifice comprises a proximal base member and a plurality of conduits arranged to receive a corresponding plurality of surgical tools. The device further comprises, for each conduit: (i) a lumen shaped for passage therethrough of a surgical tool, the lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity.

In some embodiments, at least one of the gas ports can be configured for insufflating therethrough a gas, from an external source of gas, into the body cavity, and/or at least one of the gas ports can be configured for expelling insufflated gas therethrough, and/or he respective proximally-sealed distal gas volumes of the plurality of conduits can combine to form, in combination with the body cavity, a closed gas volume.

In some embodiments, it can be that when a surgical tool is inserted into the body cavity through a respective conduit of the plurality of conduits, the distal gas volumes of both the respective conduit and at least one other conduit of the plurality of conduits remain proximally-sealed and in communication with the body cavity.

In some embodiments, it can be that when the plurality of surgical tools have passed through the respective lumens to access the body cavity, and a surgical tool of the plurality of surgical tools is withdrawn proximally from the body cavity and passes out of the respective lumen through the proximal port, the distal gas volumes of both the respective conduit and at least one other conduit of the plurality of conduits remain proximally-sealed and in communication with the body cavity.

In some embodiments, it can be that the closed gas volume includes an insufflated gas and remains closed when a surgical tool is inserted into the body cavity through a respective conduit of the plurality of conduits. In some embodiments, it can be that the closed gas volume includes an insufflated gas and remains closed when a surgical tool is removed from the body cavity and passes out of a respective lumen through the proximal port. In some embodiments, each of the lumens can be entirely surrounded by a corresponding conduit. In some embodiments, a respective longitudinal portion of one or more of the lumens can be surrounded by the proximal base member. In some embodiments, a respective longitudinal portion of one or more conduits of the plurality of conduits can be surrounded by the proximal base member.

In some embodiments, an assembly can comprise: (i) the device of any one of claims 1 to 9, (ii) for each respective gas port, a fluid conveyance in communication therewith and comprising a respective fluid valve, and (iii) a source of a gas for insufflation of the body cavity, arranged for insufflating the gas into the body cavity through at least one of the fluid conveyances. In some embodiments, at least one of the fluid conveyances can be arranged for expelling insufflated gas therethrough. In some embodiments, the respective proximally-sealed distal gas volumes of the plurality of conduits can combine to form, in combination with the body cavity, a closed gas volume that remains closed when a surgical tool is inserted into the body cavity through a respective conduit of the plurality of conduits. In some embodiments, the closed gas volume can remain closed when a surgical tool is removed from the body cavity and passes out of a respective lumen through the proximal port.

In some embodiments, the device can additionally comprise, for each conduit: a dilator, and an actuatable puncturing needle biased to a withdrawn position and arranged to pass through an aperture at a distal tip of the dilator. In some embodiments, the dilator can be configured for passage through the proximal sealing gasket, such that when the dilator is withdrawn proximally from the body cavity and passes out of the respective conduit through the proximal port, the distal gas volume of at least one other conduit of the plurality of conduits remains proximally-sealed and in communication with the body cavity. In some embodiments, it can be that the closed gas volume remains closed when the dilator is removed from the body cavity and passes out of a respective lumen through the proximal port.

In some embodiments, the biasing can be by a biasing element disposed within the conduit. In some embodiments, the puncturing needle can be arranged to be withdrawn together with the dilator. In some embodiments, the device can additionally comprise an actuation member arranged to actuate, simultaneously, the respective puncturing needles of the plurality of conduits. In some such embodiments, the actuation member can be configured for being withdrawn from the device together with the respective puncturing needles and the respective dilators of the plurality of conduits.

In some embodiments, the conduits can comprise respective tubes.

In some embodiments, the conduits can be distally beveled to form, in combination, a section of a dilator. In some embodiments, the device can additionally comprise an actuatable puncturing needle biased to a withdrawn position and arranged to pass through an aperture at a distal tip of the device. In some embodiments, the distal tip of the device is at least partly beveled to form a section of a dilator. In some embodiments, the biasing can be by one or more biasing elements disposed outside the conduits.

In some embodiments, the device can additionally comprise an actuation member arranged to actuate the puncturing needle by overcoming a mechanical resistance of the one or more biasing elements. In some embodiments, the device can additionally comprise an activation-prevention mechanism preventing actuation of the puncturing needle without an initial action that does not cause actuation. In some embodiments, the actuation member can be displaceable to expose the proximal ports for receiving the surgical tools.

In some embodiment, the access device may include a stopper member configured for preventing activation of the actuation mechanism. Specifically, the stopper member may be configured for physically blocking the actuation member from being moved into an actuated position. In accordance with a particular example, the access device can be configured to receive, at least partially, within the lumen, a piece including said stopper member preventing activation of the actuation member. In particular, the design may be such that said piece is configured for being introduced into the lumen in a step succeeding the step of activation of the puncturing needle, thereby rendering the puncturing needle deactivated for the remainder of a procedure.

In accordance with a specific example, said piece may be formed with an axial extension constituting the blocking member. The actuation member may have a drive path along which it transitions between an actuated and non-actuated positions. Correspondingly, the blocking member may be designed such that, once the piece is inserted into the lumen of the access device, the blocking member extends axially into a position crossing the drive path of the actuation member, thereby physically blocking it from transitioning into an activated position.

In addition, the extension may be designed in order to complement the design of the access device. Specifically, the extension may also constitute a portion of a fitting mechanism, configured for retaining the inserted piece within the lumen and/or attached to the access device. Thus, the extension member may have a dual purpose—both fixing the position of the piece with respect to the access device, and, at the same time, preventing the actuation member from being activated.

It should be noted that spontaneous activation of the actuation mechanism may result in the puncturing needle being activated, which, if done in an unsupervised manner, may cause damage to tissue. The solution provided by the subject matter of the present application elegantly restricts this possibility by introducing the blocking member in a step superseding the activation of the puncturing needle.

In some embodiments, the plurality of conduits can comprise exactly two conduits.

According to embodiments disclosed herein, a device for accessing a body cavity through a natural orifice comprises a proximal base member and a plurality of conduits arranged to receive a corresponding plurality of surgical tools. The device further comprises, for each conduit: (i) a lumen shaped for passage therethrough of a surgical tool, the lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity, the respective proximally-sealed distal gas volumes of the plurality of conduits combining to form, in combination with the body cavity, a closed gas volume, wherein the closed gas volume is effective to remain closed when any one or more of the following events occur: (i) a surgical tool is inserted into the body cavity through a respective conduit of the plurality of conduits, and (ii) a surgical tool is removed from the body cavity and passes out of a respective lumen through the proximal port.

In some embodiments, it can be that when the closed gas volume includes an insufflated gas, the closed gas volume is effective to remain closed when any one or more of the following events occur: (i) a surgical tool is inserted into the body cavity through a respective conduit of the plurality of conduits, and (ii) a surgical tool is removed from the body cavity and passes out of a respective lumen through the proximal port.

In some embodiments, it can be that at least one of the gas ports is configured for insufflating therethrough a gas, from an external source of gas, into the closed gas volume, and/or that at least one of the gas ports is configured for expelling insufflated gas therethrough. In some embodiments, in can be that when (i) the closed gas volume includes an insufflated gas, (ii) a surgical tool is removed from the body cavity and passes out of a respective lumen through the proximal port, and (iii) subsequent to the removal, a replacement surgical tool is inserted into the body cavity through the respective lumen, the closed gas volume is effective to retain substantially all of the insufflated gas.

According to embodiments disclosed herein, a device for accessing a body cavity through a natural orifice comprises: (a) a proximal base member; (b) a plurality of conduits arranged to receive a corresponding plurality of surgical tools for passage therethrough to the body cavity, the conduits distally beveled to form, in combination, a section of a dilator; (c) an actuatable puncturing needle biased to a withdrawn position by one or more biasing elements disposed outside the conduits and arranged to pass through an aperture at a distal tip of the device; and (d) an actuation member arranged to actuate the puncturing needle by overcoming a mechanical resistance of the one or more biasing elements.

In some embodiments, it can be that when the actuation member is released after an actuation, the puncturing needle is configured to return to the withdrawn position for storage at least during a use of the dilator to distally advance the device. According to embodiments disclosed herein, it can be that when the actuation member is released after an actuation, the puncturing needle is configured to return to the withdrawn position for storage at least during passage of the surgical tools through the plurality of conduits to access the body cavity.

In some embodiments, the device can additionally comprise an actuation-prevention mechanism preventing actuation of the puncturing needle without an initial action that does not cause actuation. In some embodiments, the actuation member is displaceable to expose the proximal ports for receiving the surgical tools.

In some embodiments, it can bet that the device comprises exactly one actuatable puncturing needle and exactly one actuation member.

In some embodiments, the distal tip can be formed integrally with the plurality of conduits. In some embodiments, the plurality of conduits can be formed together and not be separable non-destructively.

In some embodiments, the plurality of conduits can comprise exactly two conduits.

In some embodiments, a transverse cross-section of the device intercepting the plurality of conduits at any point distal to the proximal base can be such that a minimum-area circumscription of the cross-section is concave.

In some embodiments, the device can further comprise, for each conduit: (i) a lumen shaped for passage therethrough of a surgical tool, the lumen having a proximal port and a distal opening, (ii) a gas port in fluid communication with the lumen and displaced distally from the proximal port, and/or (iii) a proximal sealing gasket seated in the lumen and adapted for selectively opening and closing. The gasket can be displaced distally from the proximal port and proximally from the gas port, e.g., so as to create a respective proximally-sealed distal gas volume in communication with the body cavity. At least one of the gas ports can be configured for insufflating therethrough a gas, from an external source of gas, into the body cavity. At least one of the gas ports can be configured for expelling insufflated gas therethrough.

In some embodiments, (i) the respective proximally-sealed distal gas volumes of the plurality of conduits can combine to form, in combination with the body cavity, a closed gas volume, and/or (ii) the closed gas volume can be effective to remain closed when a surgical tool is inserted into the body cavity through a respective conduit of the plurality of conduits.

In some embodiments, the respective proximally-sealed distal gas volumes of the plurality of conduits can combine to form, in combination with the body cavity, a closed gas volume, and the closed gas volume can be effective to remain closed when a surgical tool is removed from the body cavity and passes out of a respective lumen.

In some embodiments, the closed gas volume can include an insufflated gas and can remain closed when a surgical tool is inserted into the body cavity through a respective conduit of the plurality of conduits.

In some embodiments, the closed gas volume can include an insufflated gas and can remain closed when a surgical tool is removed from the body cavity and passes out of a respective lumen through the proximal port.

A method is disclosed, according to embodiments of the present invention, for accessing a surgical site in a body cavity with multiple surgical tools through a natural orifice. The method comprises: (a) providing an access device comprising a proximal base member and a plurality of conduits, and further comprising, for each conduit: (i) a lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity; (b) inserting a distal portion of the access device through the orifice; and (c) introducing the multiple surgical tools into the body cavity through respective lumens of the access device.

In some embodiments, the proximally-sealed distal gas volumes can remain proximally-sealed and in communication with the body cavity during the introducing.

In some embodiments, the introducing can include introducing one surgical tool at a time. In some embodiments, the introducing can include introducing one or more of the multiple surgical tools in parallel.

In some embodiments, the method can additionally comprise, after the introducing of the multiple surgical tools, proximally withdrawing a surgical tool of the multiple surgical tools from the body cavity and out of the respective lumen via the proximal port. The proximally-sealed distal gas volumes can remain proximally-sealed and in communication with the body cavity.

In some embodiments, the surgical tools can comprise robotic tools, and the introducing of the surgical tools can include controlling the movements of the robotic tools.

In some embodiments, the method can additionally comprise: (i) placing an external gas source in communication with a respective gas port of the plurality of conduits via a fluid conveyance comprising a fluid valve, and/or (ii) insufflating a gas from the gas source into the body cavity. In some such embodiments, the method can additionally comprise expelling insufflated gas through a gas port of the plurality of conduits into a fluid conveyance comprising a fluid valve. In some embodiments, the method can additionally comprise: replacing a surgical tool when the closed gas volume includes an insufflated gas. The closed gas volume can be effective to remain closed when any one or more of the following events occur: (i) a surgical tool is introduced into the body cavity through a respective conduit of the plurality of conduits, and (ii) a surgical tool is removed from the body cavity and passes out of a respective lumen through the proximal port.

In some embodiments, the plurality of conduits can comprise exactly two conduits.

In some embodiments, the provided access device can include one or more previously used conduits.

In some embodiments, the access device can additionally comprise, for each conduit: a dilator, and an actuatable puncturing needle biased to a withdrawn position and arranged to pass through an aperture at a distal tip of the dilator. The biasing can be by a biasing element disposed within the conduit. In some embodiments, the method can additionally comprise: (i) actuating, simultaneously, the respective puncturing needles of the plurality of conduits, using an actuation member of the access device, (ii) forming an incision in tissue which separates the body cavity from the orifice, and/or (iii) dilating the incision by advancing the distal portion of the access device therethrough to place respective distal openings of the conduits in communication with the body cavity.

In some embodiments, the method can additionally comprise proximally withdrawing the respective dilators of the plurality of conduits from the body cavity, and/or causing the respective dilators to pass out of the conduits. The proximally-sealed distal gas volumes can remain proximally-sealed and in communication with the body cavity.

In some embodiments, the puncturing needles and the biasing elements can be withdrawn together and/or are withdrawable with the dilator. In some embodiments, the puncturing needles and actuation member(s) can be withdrawn together and/or are withdrawable with the dilator.

In some embodiments, the conduits can comprise respective tubes.

In some embodiments, the conduits can be distally beveled to form, in combination, a section of a dilator. In some embodiments, the access device can additionally comprise an actuatable puncturing needle that is biased, by one or more biasing elements disposed outside the conduits, to a withdrawn position and arranged to pass through an aperture at a distal tip of the device. The distal tip of the device can be at least partly beveled to form a section of a dilator.

In some embodiments, the method can additionally comprise: (i) actuating the puncturing needle using an actuation member arranged to overcome a mechanical resistance of the one or more biasing elements, (ii) forming an incision in tissue which separates the body cavity from the orifice, and/or (iii) dilating the incision by advancing the distal portion of the access device therethrough to place respective distal openings of the conduits in communication with the body cavity. In some embodiments, the access device can additionally comprise an activation-prevention mechanism preventing actuation of the puncturing needle without an initial action that does not cause actuation. In some embodiments, the actuation member can be displaceable to expose the proximal ports for receiving the surgical tools.

A method is disclosed, according to embodiments of the present invention, for accessing a surgical site in a body cavity with multiple surgical tools through a natural orifice. The method comprises: (a) providing an access device comprising: (i) a proximal base member, (ii) a plurality of conduits distally beveled to form, in combination, a section of a dilator, and (iii) a single actuatable puncturing needle disposed outside the conduits and biased to a withdrawn position by one or more biasing elements; (b) inserting, through the orifice, a distal portion of the access device; (c) actuating the puncturing needle using an actuation member arranged to overcome a mechanical resistance of the one or more biasing elements, wherein the actuating includes (i) passing a distal tip of the puncturing needle through an aperture at a distal tip of the device, and (ii) forming an incision in tissue which separates the body cavity from the orifice; (d) dilating the incision by distally advancing the access device therethrough to place respective distal openings of the conduits in communication with the body cavity; and (e) introducing the multiple surgical tools into the body cavity through respective conduits.

In some embodiments, it can be that when the actuation member is released after an actuation, the puncturing needle is configured to return to the withdrawn position for storage at least during the distally advancing. In some embodiments, it can be that when the actuation member is released after an actuation, the puncturing needle is configured to return to the withdrawn position for storage at least during the introducing.

In some embodiments, the access device can include an actuation-prevention mechanism preventing actuation of the puncturing needle without an initial action that does not cause actuation, and actuating includes performing the initial action that does not cause actuation. In some embodiments, the method can additionally comprise displacing the actuation member to expose the proximal ports for receiving the surgical tools.

In some embodiments, the access device can comprise exactly one actuatable puncturing needle and exactly one actuation member. In some embodiments, the plurality of conduits can comprise exactly two conduits. In some embodiments, the distal tip of the access device can be formed integrally with the plurality of conduits. In some embodiments, the plurality of conduits can be formed together and/or be not separable non-destructively.

In some embodiments, a transverse cross-section of the access device intercepting the plurality of conduits at any point distal to the proximal base can be such that a minimum-area circumscription of the cross-section is concave.

In some embodiments, it can be that (i) the access device further comprises, for each conduit: (i) a lumen shaped for passage therethrough of a surgical tool, the lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity, and/or (ii) the method additionally comprises insufflating a gas from an external source of gas through at least one of the gas ports and into the body cavity. In some embodiments, the method can additionally comprise expelling insufflated gas through at lest one of the gas ports.

In some embodiments, it can be that the respective proximally-sealed distal gas volumes of the plurality of conduits combine to form, in combination with the body cavity, a closed gas volume, and/or that the closed gas volume remains closed during the inserting.

In some embodiments, it can be that (i) the respective proximally-sealed distal gas volumes of the plurality of conduits combine to form, in combination with the body cavity, a closed gas volume, (ii) the method additionally comprises: withdrawing a surgical tool, the withdrawing including removing the surgical tool from the body cavity and causing the surgical tool to pass out of a respective lumen, and/or (iii) the closed gas volume is effective to remain closed during the withdrawing. In some embodiments, the closed gas volume can include an insufflated gas.

According to embodiments, an apparatus for accessing a body cavity through a natural orifice comprises: (a) a distal cannula assembly distally beveled to form a section of a dilator; (b) a proximal base member comprising a plurality of conduits shaped to be seated within the cannula member and receive therein a corresponding plurality of surgical tools for passage therethrough to the body cavity; and (c) an actuatable puncturing needle biased to a withdrawn position by a biasing element and arranged to pass through an aperture at a distal tip of the distal cannula assembly. The distal cannula assembly comprises an actuation member arranged to actuate the puncturing needle by overcoming a mechanical resistance of the one or more biasing elements; and the proximal base member comprises a blocking element shaped to block an actuating movement of the actuation member when the distal cannula assembly and proximal base member are joined. In some embodiments, the distal cannula assembly and proximal base member are formed to snap together when joined.

In some embodiments, when the actuation member is released after an actuation, the puncturing needle is configured to return to a withdrawn position by the biasing element. The actuation member is further configured to be restrained in said withdrawn position by the blocking element while the distal cannula assembly and proximal base member are joined.

In some embodiments, a method of accessing a surgical site in a body cavity with multiple surgical tools through a natural orifice comprises (a) providing an apparatus comprising: (i) a distal cannula assembly distally beveled to form a section of a dilator, (ii) a proximal base member comprising a plurality of conduits shaped to be seated within the cannula member and receive therein a corresponding plurality of surgical tools for passage therethrough to the body cavity, and (iii) an actuatable puncturing needle biased to a withdrawn position by a biasing element and arranged to pass through an aperture at a distal tip of the distal cannula assembly, wherein the distal cannula assembly comprises an actuation member arranged to actuate the puncturing needle by overcoming a mechanical resistance of the one or more biasing elements, the distal cannula assembly and proximal base member are formed to snap together when joined, and the proximal base member comprises a blocking element shaped to block an actuating movement of the actuation member when the distal cannula assembly and proximal base member are joined; (b) inserting, through the orifice, a distal portion of the distal cannula member; (c) actuating the puncturing needle using the actuation member, wherein the actuating includes (i) passing a distal tip of the puncturing needle through the aperture at a distal tip of the device, (ii) forming an incision in tissue which separates the body cavity from the orifice, and (iii) releasing the actuation member such that the puncturing needle is returned by the biasing element to the withdrawn position; (d) joining the proximal base member and the distal cannula member, thereby blocking an additional actuating movement of the actuation member; (e) dilating the incision by distally advancing the apparatus therethrough to place respective distal openings of the conduits in communication with the body cavity; and (f) introducing the multiple surgical tools into the body cavity through respective conduits.

In some embodiments, the joining includes causing the proximal base member and the distal cannula member to snap together

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 is a schematic top view of an access device according to embodiments of the present invention.

FIG. 2 is a schematic cross-sectional view of the access device of FIG. 1, according to embodiments of the present invention.

FIG. 3 is a schematic cross-sectional in situ view of the access device of FIG. 1 with a surgical tool introduced through the access device into a body cavity, according to embodiments of the present invention.

FIG. 4 is a schematic cross-section in situ view of the access device of FIG. 1 showing a closed gas volume including the body cavity insufflated through the access device from an external source of gas, according to embodiments of the present invention.

FIG. 5A is a schematic top view of an exemplary access device according to embodiments of the present invention.

FIG. 5B is a schematic transverse cross-section of the access device of FIG. 5A, according to embodiments of the present invention.

FIG. 6 shows a proximal portion of the access device of FIG. 5A, with the proximal base member being made transparent to show interior details of the device, according to embodiments of the present invention.

FIG. 7A is a schematic top view of an exemplary access device according to embodiments of the present invention.

FIG. 7B a schematic transverse cross-section of the access device of FIG. 7A, according to embodiments of the present invention.

FIG. 8A shows a proximal portion of the access device of FIG. 7A, with the proximal base member being made transparent to show interior details of the device, according to embodiments of the present invention.

FIG. 8B shows a proximal portion of the access device of FIG. 5A, with the proximal base member and the conduits being made transparent to show interior details of the device, according to embodiments of the present invention.

FIG. 9A is a schematic top view of an exemplary access device according to embodiments of the present invention.

FIG. 9B a schematic transverse cross-section of the access device of FIG. 9A, according to embodiments of the present invention.

FIGS. 9C and 9D show the cross-sectional view of FIG. 9B with respective examples of conduits traversing the device, according to embodiments of the present invention.

FIGS. 10A, 10B, 10C, 11A, 11B, and 11C are respective schematic transverse cross-sections of access devices having multiple conduits, according to embodiments of the present invention.

FIGS. 12A and 12B are respective schematic distal-end and top views of the access device of FIG. 5A, showing respective dilators resident in the conduits, according to embodiments of the present invention.

FIG. 12C shows the access device of FIG. 12B with the dilators removed to show details, according to embodiments of the present invention.

FIGS. 13A and 13B show the access device of FIG. 12B with respective puncturing needles actuated, respectively in top and side views, according to embodiments of the present invention.

FIGS. 14A and 14B are respective partial side and top views of the access device of FIG. 5A, according to embodiments of the present invention.

FIGS. 15A and 15B show the access device of FIGS. 14A and 14B, respectively with the puncturing needle actuated, according to embodiments of the present invention.

FIGS. 16A and 16B are respective partial side and top views of the access device of FIG. 5A, with the proximal base member and conduits made transparent to show details, according to embodiments of the present invention.

FIGS. 16C and 16D are additional view of the access device of FIG. 5A, with the actuation member removed to show detail, and with the actuation member and actuation- prevention mechanism shown, respectively, according to embodiments of the present invention.

FIG. 17 is a schematic perspective view of an element of an access device, according to embodiments of the present invention.

FIGS. 18A, 18B, 18C, 18D, 18E, 18F, 18G, and 18H show flowcharts of methods and method steps for accessing a surgical site in a body cavity with multiple surgical tools through a natural orifice, according to embodiments of the present invention.

FIGS. 19A, 19B, 19C, 19D and 19E show additional flowcharts of methods and method steps for accessing a surgical site in a body cavity with multiple surgical tools through a natural orifice, according to embodiments of the present invention.

FIGS. 20A, 20B, 20C, 20D, 21, 22, 23, and 24 are schematic exemplary in situ illustrations of selected method steps for accessing a surgical site in a body cavity with multiple surgical tools through a natural orifice, and associated procedures, according to embodiments of the present invention.

FIGS. 25A, 25B, 25C, 25D and 25E show various schematic views of an access device according to embodiments of the present invention.

FIGS. 26A and 26B illustrate a sighting assembly of the access device of FIGS. 25A-25E in open and closed states, respectively, according to embodiments of the present invention.

FIG. 27 is a view of selected components of the access device of FIGS. 25A-E, according to embodiments of the present invention.

FIGS. 28A and 28E are partial views of selected components of the access device of FIGS. 25A-E, according to embodiments of the present invention.

FIG. 29A is a schematic isometric view of an access device of the present application, shown in a non-activated and non-loaded position;

FIG. 29B is a schematic enlarged portion of a longitudinal cross-section of the access device shown in FIG. 29A;

FIG. 30A is a schematic isometric view of the access device of FIGS. 29A-29B, shown in an activated and non-loaded position;

FIG. 30B is a schematic enlarged portion of a longitudinal cross-section of the access device shown in FIG. 30A;

FIG. 31A is a schematic isometric view of the access device of FIGS. 29A-29B, shown in a non-activated, loaded position; and

FIG. 31B is a schematic enlarged portion of a longitudinal cross-section of the access device shown in FIG. 31A.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

Embodiments disclosed herein relate to access devices, including vaginal access devices and access kits for use in ‘minimally invasive’ surgical procedures requiring transvaginal introduction of surgical tools to a surgical site in the body cavity of a patient. The access device according to embodiments of the present invention acts as a multi-lumen cannula for passing tools therethrough and into the body cavity. The access device also incorporates features of a trocar, such as, for example, a puncturing needle, and a dilator for enabling advancement of the access device through an incision made by the puncturing needle and into the body cavity so as to be in communication with the surgical site. Thus, the access device is effective to reduce the number of separate elements needed for initiating the surgical procedure, which can include inserting the access kit into and through a natural body orifice, e.g., the vagina, and into the body cavity, e.g., by passing the access device through the rectouterine pouch after making an incision and advancing the access device through the incision while dilating it. Further, the access devices disclosed here include separate cannular conduits for each surgical tool, inter alia enabling replacement of tools through respective conduits without compromising sterility or losing gas pressure at the surgical site, e.g., following insufflation of the body cavity. Each tool passes through a lumen that equipped with a sealing gasket positioned to proximally seal the lumen, which is open to the body cavity. Each lumen is in communication with a gas port such that insufflation or expulsion of insufflated gas can be accomplished through any one of the conduits. The gas management regime is such that each conduit is associated with a gas port and thereby with external gas conveyances and sources for insufflation and/or expulsion.

The terms ‘access kit’ and ‘access device’ are used interchangeably herein, and mean a device (or kit, e.g., an assembly) for providing sterile access for surgical tools to a surgical site within a body cavity.

The term ‘tool’ or ‘surgical tool’ as used herein means any surgical tool deliverable to the surgical site by an articulated and/or robotic surgical arm, e.g., when installed at or attached to a distal end of the arm. The terms can be meant as including the arm or a distal portion of the arm, or the actual tool itself. Thus, a conduit shaped to receive a surgical tool means an elongated conduit through which the tool and a distal portion of a robotic surgical arm may pass unhindered.

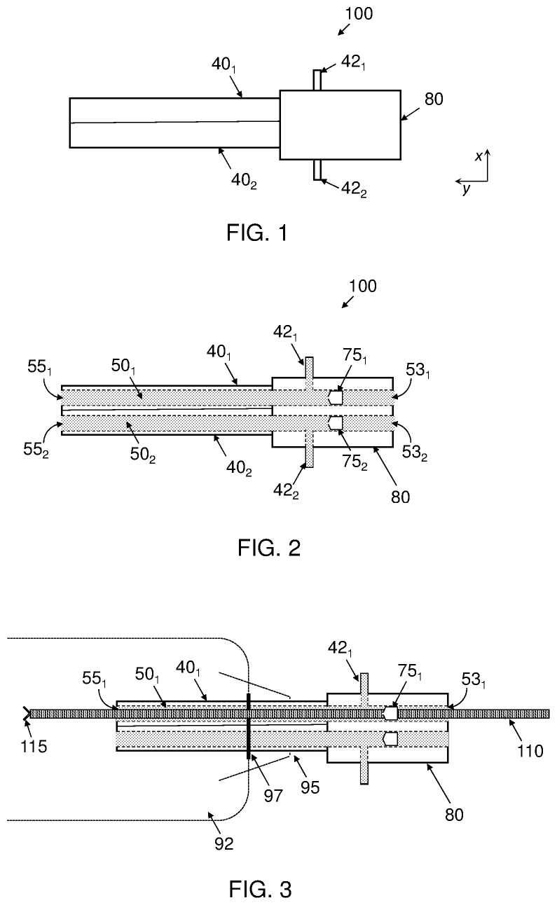

We now refer to the figures, and in particular to FIGS. 1, 2, 3 and 4. FIG. 1 is a schematic top view of an access device 100 for accessing a surgical site in a body cavity 92, with multiple surgical tools, through a natural orifice 95. The access device 100 includes a plurality of conduits 40, e.g., 40₁, 40₂, and a proximal base member 80. Examples of different conduit designs are disclosed hereinbelow. The number of conduits can be two, three, four, or more, although for some procedures, 4 may be a practical limit. In some embodiments, the plurality of conduits 40 comprises exactly two conduits 40₁, 40₂. The subscripted numbers are for convenient identification of elements in the drawings and there is no importance to be associated with, e.g., which of the two conduits is the ‘1’ conduit or the ‘2’ conduit). For convenience, all of the illustrated examples show two conduits except when illustrating conduit arrangements comprising more than two conduits.

The terms ‘distal’ and ‘proximal’ are used herein according to the common convention wherein ‘distal’ means closer to the center of the human body, or further/deeper towards or into the human body, while ‘proximal’ is the opposite direction. The convention is maintained in figures such as FIG. 1 in which the access device is not shown in situ, but rather in the abstract. The ‘distal’ end is the end that goes first and deepest into the patient's body. For the sake of clarity, the respective directions are indicated by arrow 1200 in FIG. 1, and this convention is maintained throughout the figures for all complete plan (top or bottom views) and side views of access devices.

The access device 100 of FIG. 1 is shown in cross-section in FIGS. 2, 3 and 4, the section being in an x-y plane in accordance with the x- and y-axes shown in FIG. 1.

FIGS. 2 and 3 show that for each conduit 40, the access device 100 comprises a corresponding lumen 50 that provides a tool-path from a proximal port 53 to a distal opening 55. In examples discussed hereinbelow in greater detail, a conduit 40 and a lumen 50 do not necessarily refer to the same conveyance. The term ‘conduit’ as used herein refers to a physical conveyance such as, for example, a tube, while the term ‘lumen’ is used to mean a volume contained by a ‘conduit’ or other conveyance, e.g., a different vessel. For example, a conduit 40 may end in proximity to its entry point into the proximal base member 80, while the lumen 50 extends proximally beyond the proximal end of the conduit 40, all the way to the proximal port 53 through bores in the base member 80. Thus, in some designs, each of the lumens 50 is entirely surrounded by a corresponding conduit 40, in which case a longitudinal portion of the conduit 40 may or may not pass at least partly through the proximal base member 80 and be surrounded thereby. In some designs, a longitudinal portion of a lumen 50 can be surrounded by the proximal base member 80 without the presence of an intervening portion of a conduit 40.

The tool-path is a path for passage of a surgical tool 115. FIG. 3 shows, for purpose of illustration, a single surgical tool 115 at the distal end of a surgical arm 110 which passes through the first lumen 50₁ and into the body cavity 92. FIG. 3 further illustrates the distal portion of the access device having been inserted into a natural orifice 95 and advanced through a tissue 97, e.g., a rectouterine pouch, separating the orifice 95 from a body cavity 92.

The access device 100 also comprises, for each conduit 40, a gas port 42, e.g., for insufflating gas into a body cavity 92 and/or for expelling insufflated gas therefrom. As can be seen in FIGS. 2 and 3, each gas port 42 is in fluid communication with a respective lumen 50.

The access device 100 also comprises, for each conduit, a sealing gasket 75, formed for selectively opening and closing when an object, such as, for example, a dilator shaft or robotic surgical arm, passes through it in the distal direction (or in the proximal direction if the dilator shaft or robotic surgical arm has left the gasket opened to surround the shaft or arm following the distal-ward passage of the shaft or arm, and the gasket remains closed or sealed around the shaft or arm as long as the shaft or arm is present). An example of a suitable sealing gasket is a rubber or silicone one-way valve, e.g., a duckbill valve. The sealing gasket 75 is seated in the lumen 50 and prevents fluids, such as gases, from flowing through in a proximal direction, and it effectively creates a proximally-sealed gas volume, between the gasket 75 and the distal opening 55 of the conduit 40. The proximally-sealed gas volume is open to the body cavity 92.

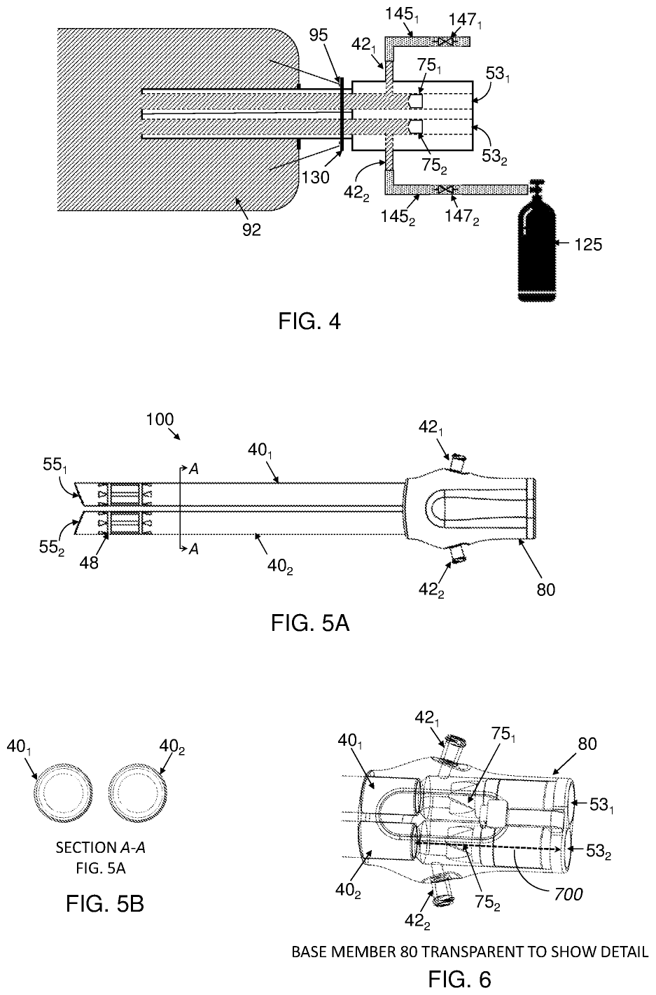

FIG. 4 further illustrates the design of the access device for maintaining a gas volume for the surgical procedure. A respective fluid conveyance 145, e.g., a tube, is connected to each of the gas ports 42. Each external fluid conveyance 145 comprises a fluid valve 147, enabling the surgeon or surgical support staff to create a closed gas volume that comprises: the portion of the volume of the lumens 50 distal of the respective sealing gaskets 75; the gas ports 42 and the portion of the volume of the external fluid conveyances up to the fluid valves 147; and the body cavity 92. This closed gas volume remains closed even when an apparatus such as a dilator shaft or a surgical tool 115, i.e., including a surgical robotic arm 110, passes through the gasket 75. When a surgical tool 115 passes proximally through the gasket 75, the design of the gasket is effective to reduce gas leakage around the tool arm to a de minimis amount or even eliminate the gas leakage. When the same tool 115 is withdrawn proximally through the gasket 75, the design of the gasket likewise continues to largely prevent, or eliminate, any leakage around the tool arm, and the gasket 75 closes again—because the ‘valve’ aspect of the gasket design is biased to close—once the tool has passed out proximally through the gasket. Thus, a single tool can be replaced during a surgical procedure without requiring any other tools to be withdrawn and reinserted, and/or without disturbing a sterile seal at the orifice (not shown in FIG. 4), while a gas pressure, e.g., an overpressure due to insufflation of a gas into the body cavity, is not substantially affected. As shown in FIG. 4, an external fluid conveyance 145 can be connected to an external gas source 125 for insufflation of gas into the body cavity 92. Similarly, insufflated gas (and other gases) can be expelled from the body cavity 92 through a gas port 42 into a corresponding external fluid conveyance 145 (and through an opened fluid valve 147).

We now refer to FIGS. 5A, 5B and 6, which illustrate an access device 100 according to a first non-limiting example. According to the first example, the conduits 40 comprise respective tubes. Visual markings 48 are shown near the distal end of each tube, inter alia, for aiding a surgeon in locating the distal end of the device 100 within the body cavity 92 using an endoscopic camera. The gas ports 42 are angled outward and proximal-ward from the proximal base member 80 for ease of connection, e.g., to distance the connection point with an external fluid conveyance from the patient and the point of entry at the orifice. The cross-section of the device 100 shown in FIG. 5B shows again that the two conduits 40 comprise separate cylindrical tubes. In the partial perspective view of FIG. 6, the proximal base member 80 is made ‘transparent’ to show details of the arrangements within the proximal base member 80. FIG. 6 shows, inter alia, that the conduits 40₁, 40₂ have proximal ends within the proximal base member 80, distal from the proximal ports 53₁, 53₂ . . . From the proximal ends of the conduits 40₁,40₂, the lumens 50 (not identified in FIG. 6) continue in the proximal direction within the volumes provided inside the proximal base member 80 until reaching the proximal ports 53₁, 53₂. The sealing gaskets 72₁, 75₂ are seated within the portions of the respective lumens 50 that are surrounded by the proximal base member 80 and not surrounded by the conduits 40. The gas ports 42₁, 42₂, as shown previously in FIGS. 3 and 4, are in fluid communication with the respective lumens 50, and are seated in the lumens 50 distally from the sealing gaskets 75.

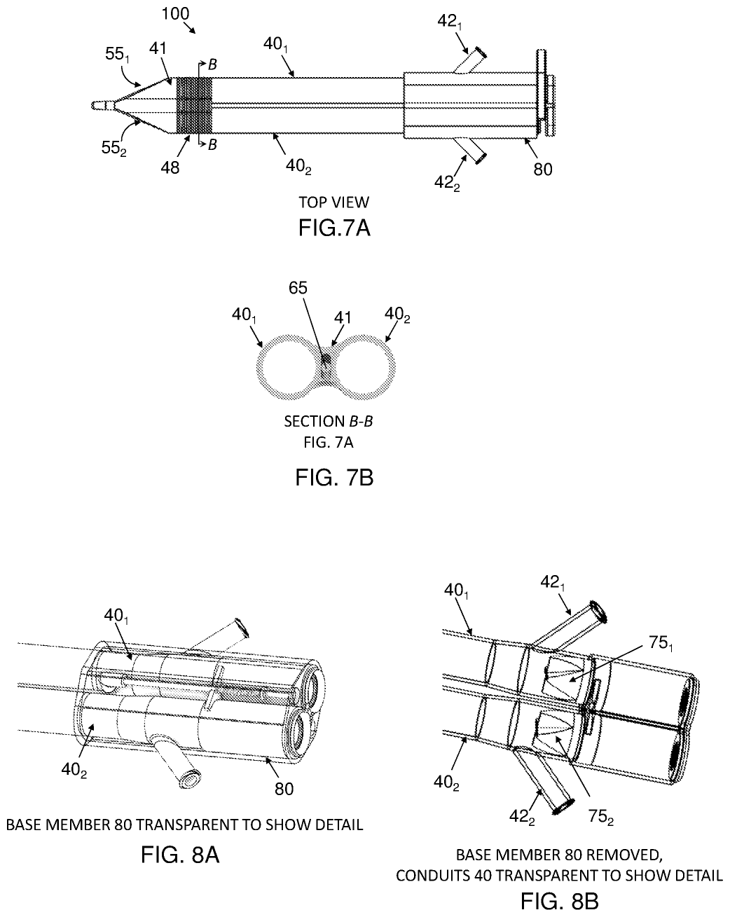

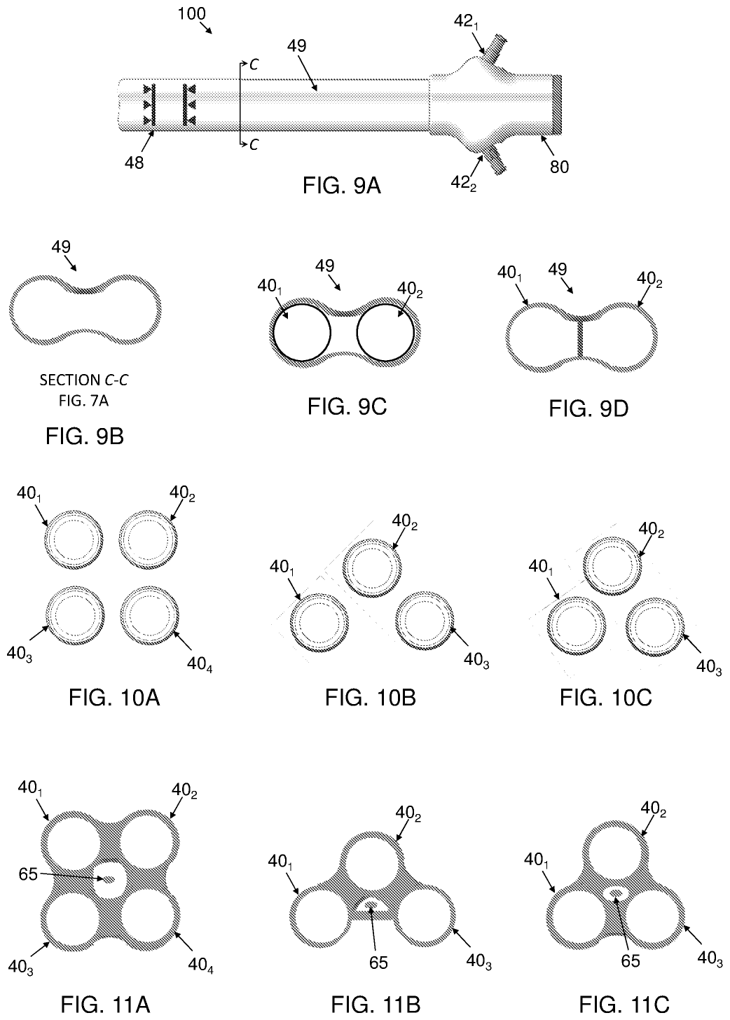

We now refer to FIGS. 7A, 7B, 8A and 8B, which illustrate an access device 100 according to a second non-limiting example. According to the second example, the conduits 40 comprise respective longitudinal portions of the device 100, but are not necessarily separable elements. In some embodiments, the conduits 40 of the second example are not non-destructively separable. Visual markings 48 are provided near the distal end of each conduit 40, similar to those in the first example. Also similar to the first example, the gas ports 42 are angled outward and proximal-ward from the proximal base member 80. The cross-section of the device 100 shown in FIG. 7B shows a puncturing needle 65, which is further discussed in connection with FIGS. 15A and 15B below, disposed outside (and between) the conduits 40₁, 40₂, as well as a conduit ‘yoke’ portion 41 bridging the conduits 40. In some designs according to the second example, the yoke portion 41 can longer, e.g., extending proximally and even up to meeting the proximal base member 80, or shorter. Respective partial perspective views in FIGS. 8A and 8B show details of the inner arrangements of the proximal base member 80. In FIG. 8A, the proximal base member 80 has been made transparent, and it is possible to see that the conduits 40₁, 40₂ according to the second design continue proximally all the way to the proximal ports 53₁, 53₂. In FIG. 8B, the proximal base member 80 is removed and the conduits 40₁, 40₂ have been made transparent, to show the sealing gaskets 72₁, 75₂ seated within the portion of the respective lumens 50 (not identified in FIGS. 8B) that are directly surrounded by the conduits 40₁, 40₂. The gas ports 42₁, 42₂, as shown previously in FIGS. 3 and 4, are in fluid communication with the respective lumens 50, and are seated in the lumens 50 distally from the sealing gaskets 75. In another variation of the second example (not shown), the conduits 40₁, 40₂ continue proximally past the gaskets 75₁, 75₂ but end before the proximal ports 53₁, 53, and the proximal ends of the lumens 50 are therefore surrounded only by the proximal base member 80 and not by the conduits 40₁, 40₂.

We now refer to FIGS. 9A, 9B, 9C and 9D, which illustrate an access device 100 according to a third non-limiting example. According to the third example, an elongated distal section 49 extends distally from the proximal base member 80. The cross-section of FIG. 9B reveals an elongated distal section 49 that is not necessarily divided into conduits. FIG. 9C shows a use case of the third example in which conduits 40, e.g., tubes, are inserted into the elongated distal section 49. FIG. 9D shows a variant of the third-example design in which the elongated distal section 49 is pre-divided into conduits.

It can be desirable for a cross-section of an access device, and in particular the distal cross section, to be of minimal size, at least because a minimal cross-section means that disturbance, and possible damage, to tissue surrounding the orifice and tissue separating the orifice from the body cavity, is limited. Additionally, it may be necessary to introduce another instrument to the body cavity, i.e., other than the surgical tools introduced by surgical robotic arms. A non-limiting example of such an instrument is a uterine manipulator. In order to reduce the combined cross-section of the access device and the additional instrument, it can be desirable to make the distal cross-section of the access device concave. The cross-sections shown in FIGS. 5B, 7B, and 9B-D are designed to be concave, in that a transverse cross-section of the access device intercepting the plurality of conduits at any point distal to the proximal base is such that a minimum-area circumscription of the cross-section is concave. Accordingly, the combined cross-section together with the additional instrument is smaller than it would be if the access-device cross-section were not concave, as is the case in prior-art cannulas accommodating multiple surgical arms. The smaller combined cross-section makes the assemblage more easily manipulable through a transvaginal sealing platform placed at the entrance to the orifice for sterility and gas containment, such as, for example, a GelPOINT® V-Path Transvaginal Access Platform available commercially from Applied Medical of California, USA.

The preceding figures have all shown access devices 100 comprising exactly two conduits 40, despite the fact that an access device 100, as stated hereinabove, can include more than two conduits, e.g., three conduits or four conduits. FIGS. 10A-10C show exemplary cross-sections of access devices 100 comprising more than two conduits 40, in accordance with the first example of FIGS. 5A-B in which each of the conduits 40 comprises a separate tube. FIGS. 10B and 10C show two different design options for an access device comprising three conduits, where the access device of FIG. 10B is designed to provide adjacent space for an additional instrument outside the conduits, and in the case of needing to introduce the additional instrument to the body cavity, FIG. 10B might afford a smaller combined cross-section than would FIG. 10C. FIGS. 11A-11C show similar multi-conduit designs according to the second example (of FIGS. 7A-B) in which the conduits are not necessarily separate and separable tubes. Each of FIGS. 11A-C shows a single puncturing needle 65 for each of the illustrated access devices 100.

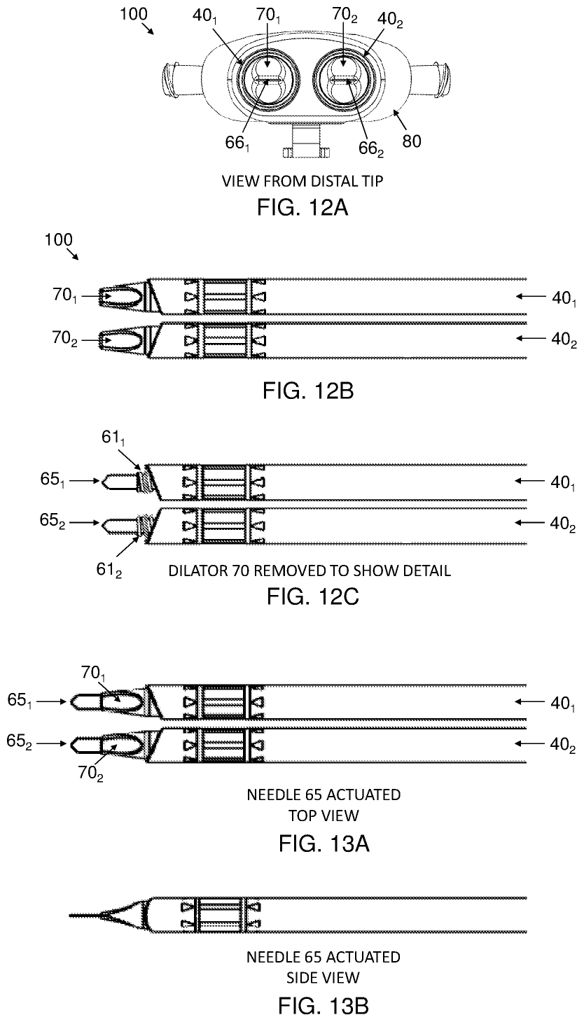

Selected additional features of the access device 100 of the first example illustrated in FIGS. 5A-B are illustrated in FIGS. 12A-C and 13A-B. For access devices 100 according to the first example, the device 100 additionally includes, for each conduit 40, an actuatable puncturing needle 65 and a dilator 70. The puncturing needle 65 is configured for use, inter alia, in making an incision in a tissue 97, e.g., a rectouterine pouch, separating the orifice 95 from a body cavity 92. The dilator 70 is configured for use, inter alia, in dilating the incision to advance the distal portion of the access device 100 into the body cavity 92.

The dilators 70 and puncturing needles 65 are either resident in the conduits 40 before a surgical operation is initiated, or inserted into the conduits 40 at the beginning of the surgical operation, for example after the access device is inserted into the orifice 95. FIG. 12A shows an end view (looking proximally) centered around the distal end of the access device 100, where the distal tips of the respective dilators 70₁, 70₂ are visible. FIG. 12B is a partial top view of the same access device 100, showing a distal portion of the access device 100. An aperture 66 at the distal tip of each dilator 70 is provided for passing a respective puncturing needle 65₁, 65₂ therethrough (See, for example, FIG. 13A). The dilator 70 is attached to, or integral with, a dilator shaft (not shown) that extends proximally through the proximal port 53 for later withdrawal of the dilator 70 to allow the surgical arms 110 to use the same conduits to access the surgical site in the body cavity 92.

FIG. 12C shows the distal portion of FIG. 12B with the dilators 70 removed to show the puncturing needles 65 within. The puncturing needles 65 are biased, e.g., by biasing element 61, to a withdrawn position proximal to the apertures 66 at the distal tips of the dilators 70. An example of a suitable biasing element 61 is a spring, as shown in FIG. 12C. In embodiments, the puncturing needles 65 of the access device 100 are simultaneously actuatable by an actuation member (not shown) arranged to overcome the mechanical resistance of the biasing elements 61 when actuated, e.g., pushed or pressed by a surgeon's thumb. FIGS. 13A and 13B are, respectively, partial top and side views of the access device of FIGS. 12A-B after the puncturing needles 65 have been actuated and pushed forward through the respective distal apertures 66.

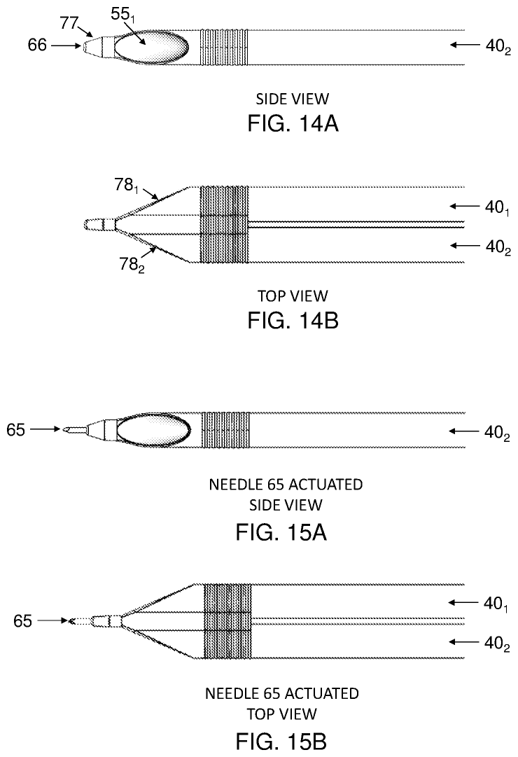

Selected additional features of the access device 100 of the second example (as shown in FIGS. 7A-B) are illustrated in FIGS. 14A-C, 15A-B and 16A-D. For access devices 100 according to the second example, the device 100 additionally includes an actuatable puncturing needle 65 and a dilator 70, e.g., a single needle 65 and a single dilator 70. The dilator 70 is ‘built in’ in the design of the access device 100 and does not necessarily require a separate dilator. As seen in the side view of a distal portion of the access device 100 in FIG. 14A, the distal tip of the access device 100 is at least partly beveled to form a first section of the dilator 70. The top view in FIG. 14B shows that the conduits 40 are distally beveled to form, in combination, a second section of the dilator 70. The beveling of the first and second sections of the dilator 70 are illustrated as being beveled in different planes, e.g., orthogonal to each other, but in some designs they can be beveled along the same or parallel planes. An aperture 66 is provided at the distal tip 77 for passing a puncturing needle 65 therethrough. In contrast to the first example design, the second example design enables the use of a single puncturing needle 65 and a single dilator 70 for the multiple conduits 40. FIGS. 15A and 15B are, respectively, partial top and side views of the access device of FIGS. 14A-B after the puncturing needle 65 has been actuated and thrust forward through the distal aperture 66.

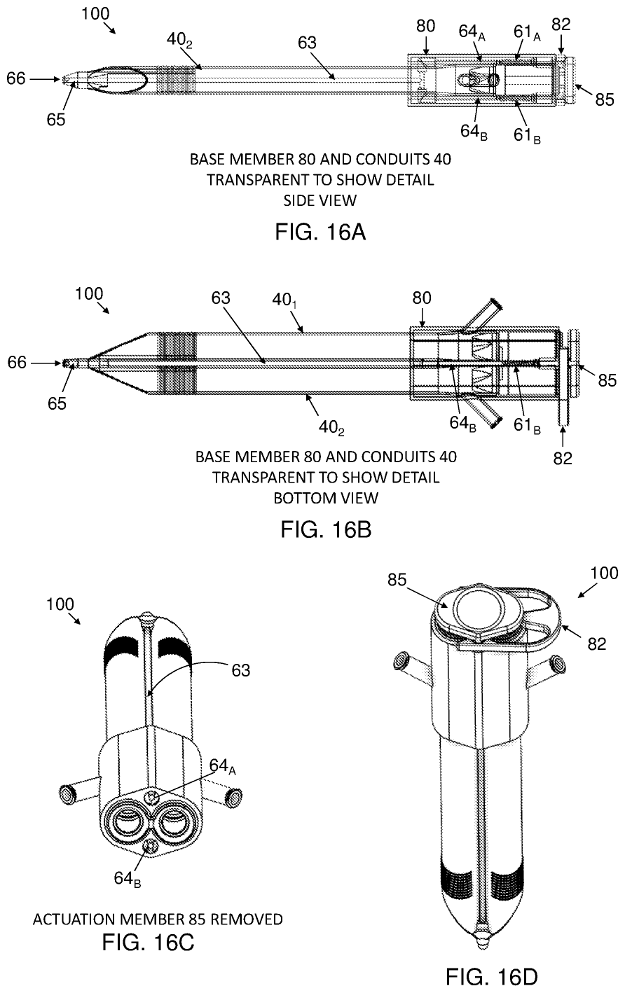

Selected additional features of the access device 100 according to the second example, and in particular of the actuation of the puncturing needle 65, are illustrated in FIGS. 16A-D. FIG. 16A is a schematic side view of an access device 100 according to the second example, as illustrated, e.g., in FIGS. 7A-B, and FIG. 16B is a corresponding top view. In both figures, the proximal base member 80 and conduits 40 are rendered as transparent to show details. The single puncturing needle 65 is disposed outside the conduits 40 and biased to a withdrawn position by one or more biasing elements 61—in the non-limiting example of FIGS. 16A-B, two biasing elements 61_{A}, 61_{B}. (The convention of using numeric subscripts herein is reserved for multiple instances of elements corresponding to multiple conduits. In this case, the two biasing elements are ‘top’ and ‘bottom’ relative to the x-y axes of FIG. 1, and do not correspond to conduits.) An actuation member 85 is provided for actuating the puncturing needle. The biasing elements 61 are yoked together by force-transfer rods 64_{A}, 64_{B} which are in communication both with the actuation member 85 and with the common force-transfer rod 63, which in turn is in mechanical communication with the punctuating needle 65. The illustrated arrangement of springs 61, multiple force-transfer rods 64_{A}, 64_{B}, 63 (also shown in FIG. 16C where the actuation member 85 is removed to show details) and a needle element 65 that is not necessarily part of the common force-transfer rod 63, is but one design option for accomplishing the transfer of force through the actuation member 85 to overcome the mechanical resistance of the (one or more) biasing elements 61 and pass at least a distal tip of the puncturing needle 65 through the aperture 66 at the distal tip 77 of the access device 100. In the instant example, the actuating is accomplished by pushing or pressing the actuation member 85.

An actuation-prevention mechanism 82 is provided to keep the actuating member 85 from being pressed to actuate the puncturing needle 65 when not required or desired, e.g., before or after it being required. The specific example of an actuation-prevention mechanism 82 shown, e.g., in FIGS. 16B and 16D is a simple sliding spacer that doesn't allow the actuation member 85 to be depressed when present. Any actuation-prevention mechanism 82 is suitable as long as it does not allow the punctuating needle 65 to be actuated without an initial action that does not cause actuation. In embodiments, the actuating of the needle 65 includes first performing the initial action that does not cause actuation, e.g., sliding the actuation-prevention mechanism 82 out of the way, and then performing the action, e.g., pushing or pressing the actuation mechanism to cause the actuation. In some embodiments, when the access device 100 is disposed in situ with a distal portion thereof inserted through the natural orifice 95 and contacting the tissue 97 which separates the body cavity 92 from the orifice 95, the actuating includes making an incision in said tissue. In practice, the incision can be made by, or as part of, the actuating of the puncturing needle 65, or can be made subsequent to the actuation when the needle 65 is extended distally through the distal-tip aperture 66. In some embodiments, the actuation member 85 must remain depressed or otherwise actuated to use the latter approach, since the biasing element(s) 61 will have caused the needle to withdraw proximally back to its initial withdrawn position in the absence of the continued depression or other actuation of the actuation member 85. In some embodiments, the actuation member 85 is displaceable, e.g., slidable, removable, foldable, etc., to expose the proximal ports for receiving the surgical tools

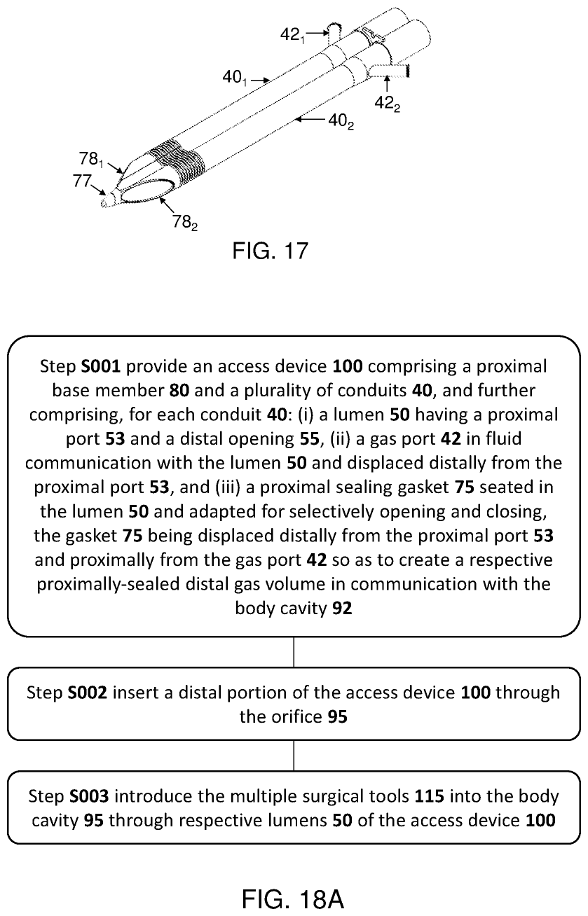

We now refer to FIG. 17. In embodiments, the distal tip 77 of the access device 100 is formed integrally with the plurality of conduits 40. In some embodiments, the plurality of conduits 40 are formed together and are not non-destructively separable. Both of these features are present in the example of FIG. 17.

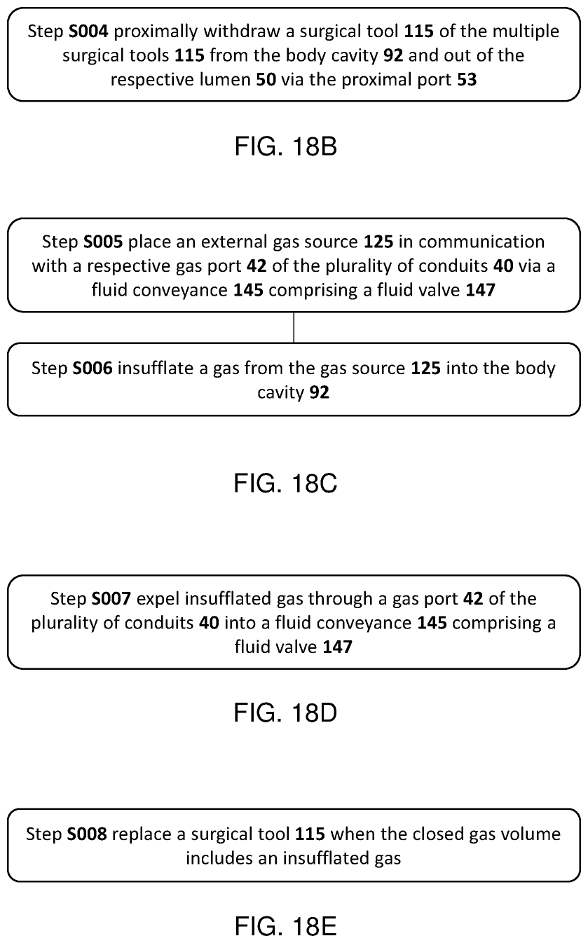

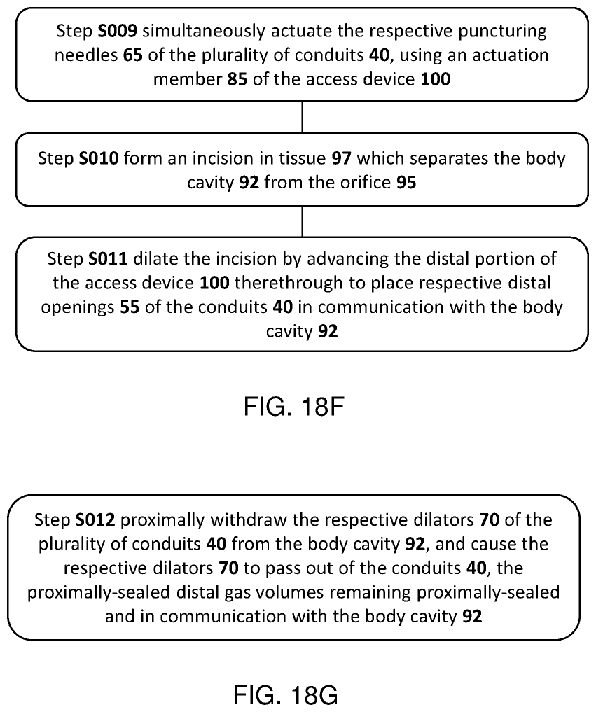

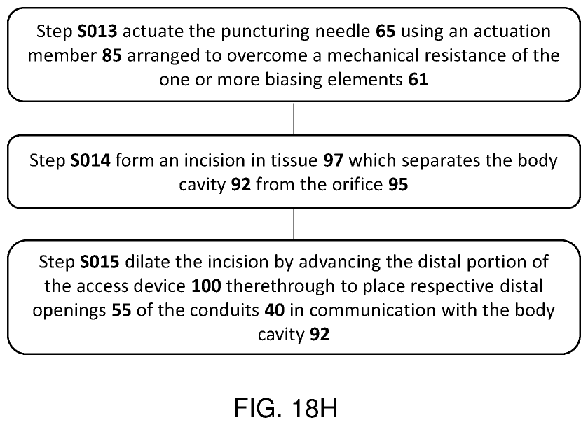

Referring now to FIG. 18A, a method is disclosed for accessing a surgical site in a body cavity 92 with multiple surgical tools 115 through a natural orifice 95. As illustrated by the flowchart in FIG. 18A, the method comprises at least the three method steps S001, S002, and S003:

Step S001 includes: providing an access device 100 comprising a proximal base member 80 and a plurality of conduits 40, and further comprising, for each conduit 40: (i) a lumen 50 having a proximal port 53 and a distal opening 55, (ii) a gas port 42 in fluid communication with the lumen 50 and displaced distally from the proximal port 53, and (iii) a proximal sealing gasket 75 seated in the lumen 50 and adapted for selectively opening and closing, the gasket 75 being displaced distally from the proximal port 53 and proximally from the gas port 42 so as to create a respective proximally-sealed distal gas volume in communication with the body cavity 92. The access device 100 can be any one of the access devices 100 disclosed in this specification, including, without limitation, access devices 100 according to any of the first, second and third examples of access-device design. In some embodiments, the access device 100 provided in Step S001 includes one or more previously used conduits 40, e.g., recycled and/or sterilized reusable conduits 40.

In some embodiments, e.g., embodiments in which the access device 100 provided in Step S001 is designed according to the first example (of FIGS. 5A-B), the access device provided in Step S001 additionally comprises, for each conduit 40: a dilator 70, and an actuatable puncturing needle 65 biased to a withdrawn position and arranged to pass through an aperture 66 at a distal tip of the dilator 70, the biasing being by a biasing element 61 disposed within the conduit 40.

In some embodiments, e.g., embodiments in which the access device 100 provided in Step S001 is designed according to the second example (of FIGS. 7A-B, etc.), the access device 100 additionally comprises an actuatable puncturing needle 65 that is biased, by one or more biasing elements 61 disposed outside the conduits 40, to a withdrawn position and arranged to pass through an aperture 66 at a distal tip 77 of the access device 100. According to embodiments, the distal tip 77 of the device is at least partly beveled to form a section of a dilator 70. In some embodiments, the access device 100 additionally comprises an activation-prevention mechanism 82 preventing actuation of the puncturing needle 65 without an initial action that does not cause actuation.

Step S002 includes: inserting a distal portion of the access device 100 through the orifice 95.

Step S003 includes: introducing the multiple surgical tools 115 into the body cavity 95 through respective lumens 50 of the access device 100. In some embodiments, wherein the proximally-sealed distal gas volumes remain proximally-sealed and in communication with the body cavity 92 during Step S003. In some embodiments, Step S003 includes introducing one surgical tool 115 at a time. In some embodiments, Step S003 includes introducing one or more of the multiple surgical tools 115 in parallel, e.g., at least partly simultaneously, i.e., such that the introduction of a tool 115 at least partly overlaps the introduction of a second tool 115. In some embodiments of the method, the surgical tools 115 comprise robotic tools, and Step S003 includes controlling the movements of the robotic tools.

In some embodiments, the method additionally comprises method Step S004, illustrated by the flowchart in FIG. 18B.

Step S004 includes: proximally withdrawing a surgical tool 115 of the multiple surgical tools 115 from the body cavity 92 and out of the respective lumen 50 via the proximal port 53. In embodiments, Step S004 is performed after Step S003. During the performance of Step S004, the proximally-sealed distal gas volumes remain proximally-sealed and in communication with the body cavity 92.

In some embodiments, the method additionally comprises method Steps S005 and S006, illustrated by the flowchart in FIG. 18C.

Step S005 includes: placing an external gas source 125 in communication with a respective gas port 42 of the plurality of conduits 40 via a fluid conveyance 145 comprising a fluid valve 147.

Step S006 includes: insufflating a gas from the gas source 125 into the body cavity 92.

In some embodiments, the method additionally comprises method Step S007, illustrated by the flowchart in FIG. 18D.

Step S007 includes: expelling insufflated gas through a gas port 42 of the plurality of conduits 40 into a fluid conveyance 145 comprising a fluid valve 147.

In some embodiments, the method additionally comprises Step S008, illustrated by the flowchart in FIG. 18E.

Step S008 includes: replacing a surgical tool 115 when the closed gas volume includes an insufflated gas. In embodiments, the closed gas volume is effective to remain closed during Step S008 when the surgical tool 115 is introduced into the body cavity 92 through a respective conduit 40 of the plurality of conduits 40, and/or when the surgical tool 115 is removed from the body cavity 92 and passes out of a respective lumen 50 through the proximal port 53.

In some embodiments, e.g., embodiments in which the access device 100 provided in Step S001 is designed according to the first example, the method additionally comprises Steps S009, S010, and S011, illustrated by the flowchart in FIG. 18F.

Step S009 includes: simultaneously actuate the respective puncturing needles 65 of the plurality of conduits 40, using an actuation member 85 of the access device 100.

Step S010 includes: forming an incision in tissue 97 which separates the body cavity 92 from the orifice 95.

Step S011 includes: dilating the incision by advancing the distal portion of the access device 100 therethrough to place respective distal openings 55 of the conduits 40 in communication with the body cavity 92.

In some embodiments, e.g., embodiments in which the access device 100 provided in Step S001 is designed according to the first example, the method additionally comprises Step S012, illustrated by the flowchart in FIG. 18G.

Step S012 includes: proximally withdrawing the respective dilators 70 of the plurality of conduits 40 from the body cavity 92, and causing the respective dilators 70 to pass out of the conduits 40. In embodiment, the proximally-sealed distal gas volumes remain proximally-sealed and in communication with the body cavity 92. In some embodiments, the puncturing needles 65 and the biasing elements 61 can be withdrawn together and/or withdrawable with the dilators 70.

In some embodiments, e.g., embodiments in which the access device 100 provided in Step S001 is designed according to the second example, the method additionally comprises Steps S013, S014, and S015, illustrated by the flowchart in FIG. 18H.

Step S013 includes: actuating the puncturing needle 65 using an actuation member 85 arranged to overcome a mechanical resistance of the one or more biasing elements 61.

Step S014 includes: forming an incision in tissue 97 which separates the body cavity 92 from the orifice 95.

Step S015 includes: dilating the incision by advancing the distal portion of the access device 100 therethrough to place respective distal openings 55 of the conduits 40 in communication with the body cavity 92.

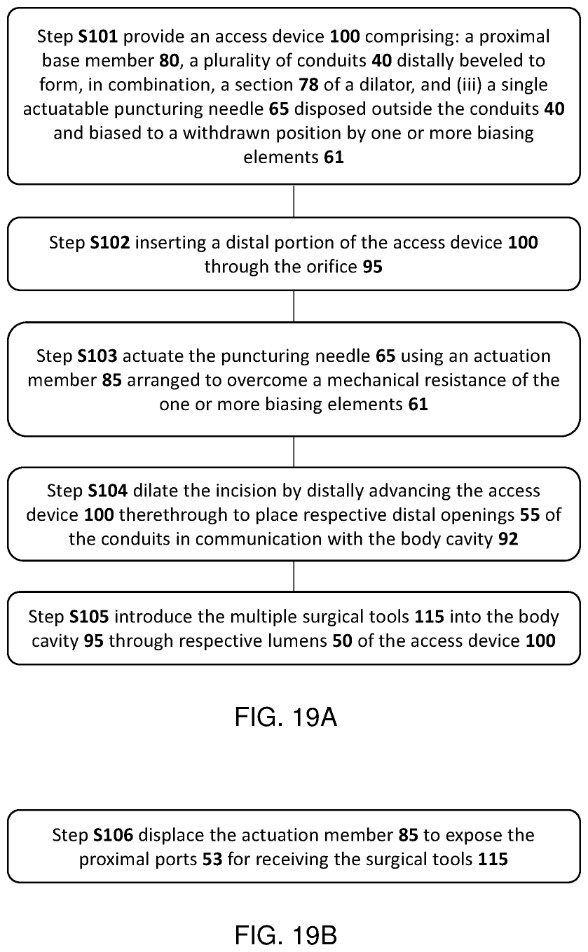

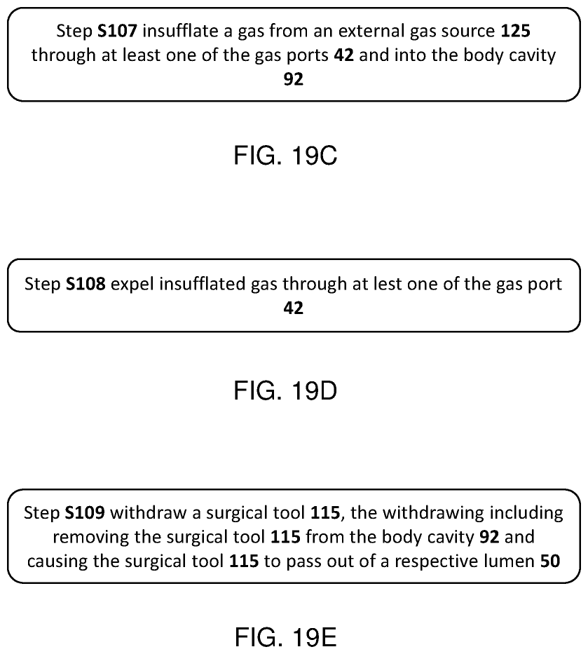

Referring now to FIG. 19A, a method is disclosed for accessing a surgical site in a body cavity 92 with multiple surgical tools 115 through a natural orifice 95. As illustrated by the flowchart in FIG. 19A, the method comprises at least the five method steps S101, S102, S103, S104, and S105:

Step S101 includes: providing an access device 100, e.g., an access device 100 designed in accordance with the second example, comprising: a proximal base member 80, a plurality of conduits 40, e.g., exactly two conduits 40₁, 40₂, distally beveled to form, in combination, a section 78 of a dilator 70, e.g., of a single dilator of the access device 100 and (iii) an actuatable puncturing needle 65, e.g., a single actuatable puncturing needle 65, disposed outside the conduits 40, e.g., between the conduits 40, and biased to a withdrawn position by one or more biasing elements 61. An example of a suitable access device 100 for performing Step S101 is the access device 100 of FIGS. 7A-B, i.e., an access device 100 according to the second design example. In some embodiments, the plurality of conduits 40 are formed together and are not non-destructively separable from each other. In embodiments, the access device is shaped such that a transverse cross-section of the access device 100 intercepting the plurality of conduits 40 at any point distally displaced from the proximal base 80, e.g., one or more of the cross-sections shown in FIGS. 5B, 7B, 9B-D, 10A-C and/or 11A-C, is such that a minimum-area circumscription of the cross-section is concave.

Step S102 includes: inserting a distal portion of the access device 100 through the orifice 95.

Step S103 includes: actuating the puncturing needle 65 using an actuation member 85 arranged to overcome a mechanical resistance of the one or more biasing elements 61. In embodiments, Step S103 includes passing a distal tip of the puncturing needle 65 through an aperture 66 at a distal tip 77 of the device 100, and forming an incision in tissue 97 which separates the body cavity 92 from the orifice 95. In some embodiments, the access device 100 includes an actuation-prevention mechanism 82 preventing actuation without an initial action that does not cause actuation, and Step S103 includes performing that initial action before performing the actuating. In some embodiments, the distal tip 77 of the access device 100 is formed integrally with the plurality of conduits 40.

Step S104 includes: dilating the incision by distally advancing the access device 100 therethrough to place respective distal openings 55 of the conduits in communication with the body cavity 92. In some embodiments, when the actuation member 85 is released after an actuation, e.g., the actuation of Step S103, the puncturing needle 65 is configured to return to the withdrawn position for storage, at least during Step S104.

Step S105 includes: introducing the multiple surgical tools 115 into the body cavity 95 through respective lumens 50 of the access device 100. In some embodiments, when the actuation member 85 is released after an actuation, e.g., the actuation of Step S103, the puncturing needle 65 is configured to return to the withdrawn position for storage, at least during Step S105.

In some embodiments, the method additionally comprises Step S106, illustrated by the flowchart in FIG. 19B.

Step S106 includes: displacing the actuation member 85 to expose the proximal ports 53 for receiving the surgical tools 115.

In some embodiments, the access device 100 further comprises, for each conduit: (i) a lumen 50 shaped for passage therethrough of a surgical tool 115, the lumen having a proximal port 53 and a distal opening 55, (ii) a gas port 42 in fluid communication with the lumen 50 and displaced distally from the proximal port 53, and (iii) a proximal sealing gasket 75 seated in the lumen 50 and adapted for selectively opening and closing, the gasket 75 being displaced distally from the proximal port 53 and proximally from the gas port 42 so as to create a respective proximally-sealed distal gas volume in communication with the body cavity 92, and the method additionally comprises Step S107, illustrated by the flowchart in FIG. 19C. In some embodiments, the respective proximally-sealed distal gas volumes of the plurality of conduits 40 combine to form, in combination with the body cavity 92, a closed gas volume, and the closed gas volume remains closed during Step S105.

Step S107 includes: insufflating a gas from an external source gas source 125 through at least one of the gas ports 42 and into the body cavity 92.

In some embodiments, e.g., embodiments in which the method comprises Step S107, the method additionally comprises Step S108, illustrated by the flowchart in FIG. 19D.

Step S108 includes: expelling insufflated gas through at lest one of the gas port 42.

In some embodiments, the respective proximally-sealed distal gas volumes of the plurality of conduits 40 combine to form, in combination with the body cavity 92, a closed gas volume, the method additionally comprises Step S109, which is illustrated in FIG. 19E, and the closed gas volume is effective to remain closed during Step S109.

Step S109 includes: withdrawing a surgical tool 115, which includes removing the surgical tool 115 from the body cavity 92 and causing the surgical tool 115 to pass out of a respective lumen 50.

The steps of any of the methods disclosed hereinabove can be performed in any order deemed practical. In some embodiments, not all of the steps are required or performed.

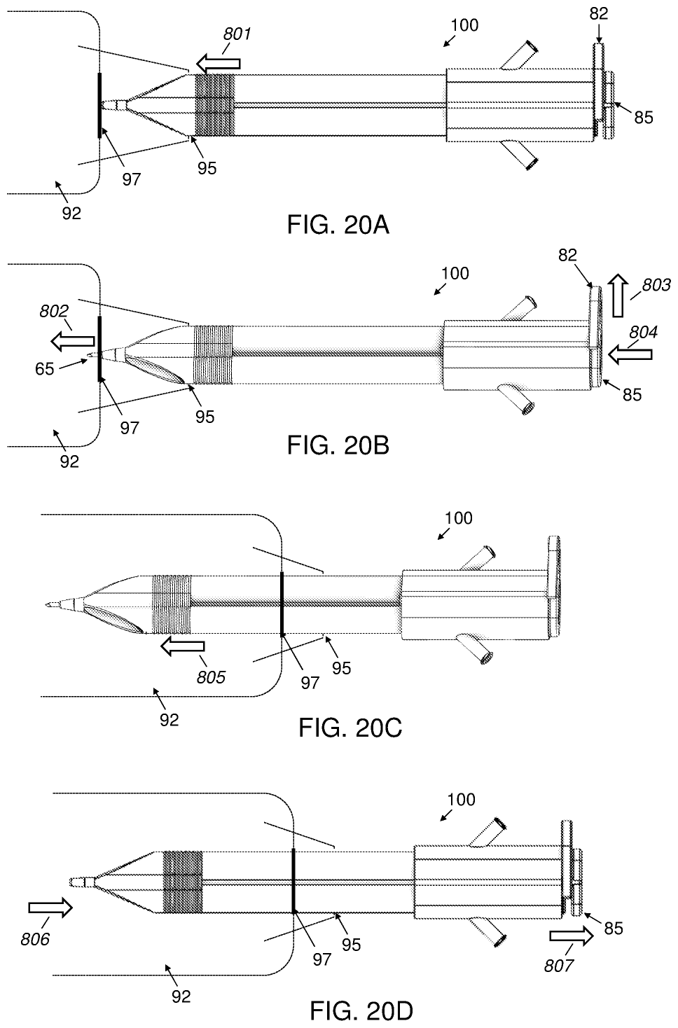

We now refer to FIGS. 20A, 20B, 20C, and 20D, which, inter alia, illustrate the performance of selected method steps of the methods disclosed hereinabove. FIGS. 20A-D illustrate the method steps using an access device 100 according to the second design example (of, e.g., FIGS. 7A-B) but access devices of any of the other examples could be used for illustrative purposes, mutatis mutandis.

FIG. 20A illustrates, inter alia, the performance of either one of Steps S002 and S102, each of which includes inserting a distal portion of the access device 100 through the natural orifice 95. The arrow marked 801 indicates the motion of the access device 100 being inserted through/into the orifice 95.

FIG. 20B illustrates, inter alia, the performance of Step S103, which includes actuating the puncturing needle 65 using an actuation member 85 arranged to overcome a mechanical resistance of the one or more biasing elements 61. According to some embodiments, the arrow marked 802 indicates the motion of the actuated needle 65 extending distally from the distal tip 77 of the access device 100. According to some other embodiments, the arrow marked 802 indicates the motion of the actuated needle 65 extending distally from the distal tip 77 of the access device 100 and making an incision in the tissue 97 separating the body cavity 92 from the orifice 95. In some embodiments, the incision is made by actuating the needle 65 while the access device is held against the tissue 97, and in some embodiments, the incision is made after actuating the needle 65. Arrow 803 indicates the motion of the actuation-prevention mechanism 82 being moved out of the way of the actuation mechanism 85 in an initial action that does not itself cause actuation, and the arrow 804 indicates the motion of the actuation itself, e.g., the pressing or pushing of the actuation mechanism 85. If FIG. 20B had been drawn with an access device 100 according to the first example, the figure would show the actuation of multiple needles 65, and would illustrate the performance of Steps S009 and S010.

FIG. 20C illustrates, inter alia, the performance of Step S104, which includes dilating the incision by distally advancing the access device 100 therethrough to place respective distal openings 55 of the conduits in communication with the body cavity 92. Arrow 805 indicates the continued motion of the distal advancing after the dilation. If FIG. 20C had been drawn with an access device 100 according to the first example, the figure would show dilation by multiple dilators 70, and would illustrate the performance of Steps S011.

FIG. 20D illustrates, inter alia, the release of the actuation member 85 after an actuation, e.g., the actuation of Step S103, at which point the puncturing needle 65 returns to the withdrawn position for storage, as indicated in FIG. 20D by arrow 806. In some embodiments, the needle 65 remains stored in the withdrawn position at least during Step S104 and/or S105.

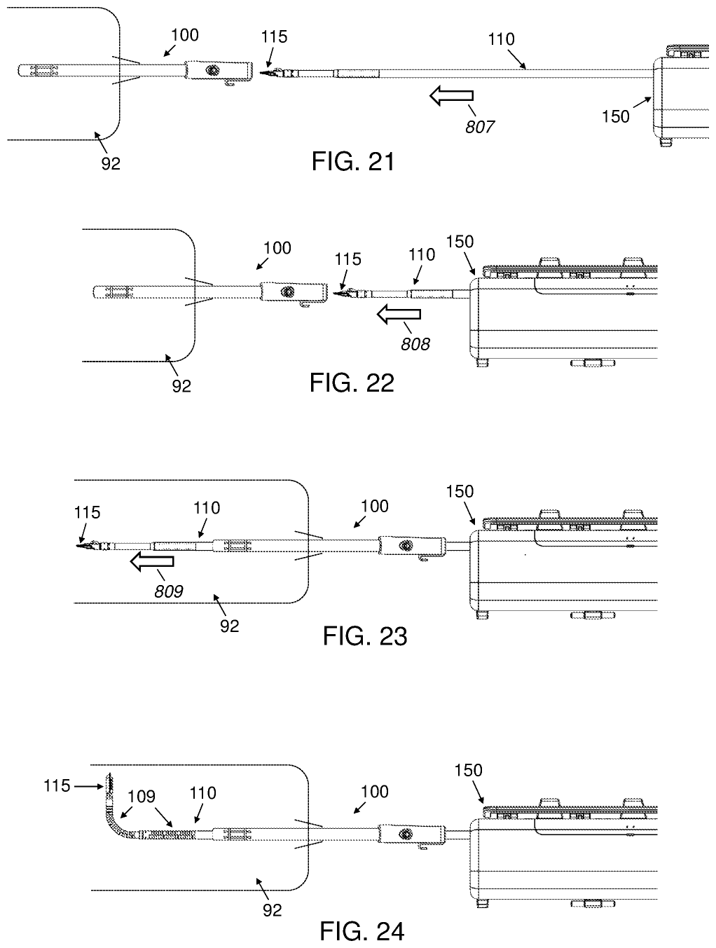

We now refer to FIGS. 21A, 22, 23, and 24, which illustrate, inter alia, the performance of selected method steps of the methods disclosed hereinabove. FIGS. 21, 22, 23, and 24 illustrate the method steps using an access device 100 according to the first design example (of, e.g., FIGS. 5A-B) but access devices of any of the other examples could be used for illustrative purposes, mutatis mutandis. The access device 100 is shown in a side view, such that only a single surgical arm 110 and surgical tool 115 is visible in the figures.

FIG. 21 shows an exemplary surgical robot 150 comprising a plurality of robot surgical arms 110. The robot 150, as indicted by arrow 807, is moved to be near an access device which is in a fixed position for a surgical procedure, e.g., following the performance Step S102. The incision and dilation have already occurred before the ‘snapshot’ view of FIG. 21, meaning that Steps S013, S014 and S015 have already been performed. In addition, it can be seen, or at least assumed that Step S012 has already been performed, and the dilator 70 and puncturing needle 65 have already been proximally withdrawn so as to evacuate the lumens 50 of the access device 100.

FIG. 22 illustrates a variation in the function of the surgical robot 150, in which the arm(s) 110 move linearly independently, i.e., arrow 808 in FIG. 22 indicates the motion of the surgical arm(s) 110 and not of the surgical robot 150.

FIG. 23 illustrates the performance of Step S003, as well as Step S105 for access devices 100 of the second design. Both of the steps include introducing the multiple surgical tools 115 into the body cavity 95 through respective lumens 50 of the access device 100, as indicated by arrow 809.

FIG. 24 illustrates, as part of a surgical procedure, the bending of a robotic surgical arm 110. The illustrated arm 110 comprises two bendable sections 99, and only one of the sections (the more distal of the two sections) is shown as being bent in FIG. 24.

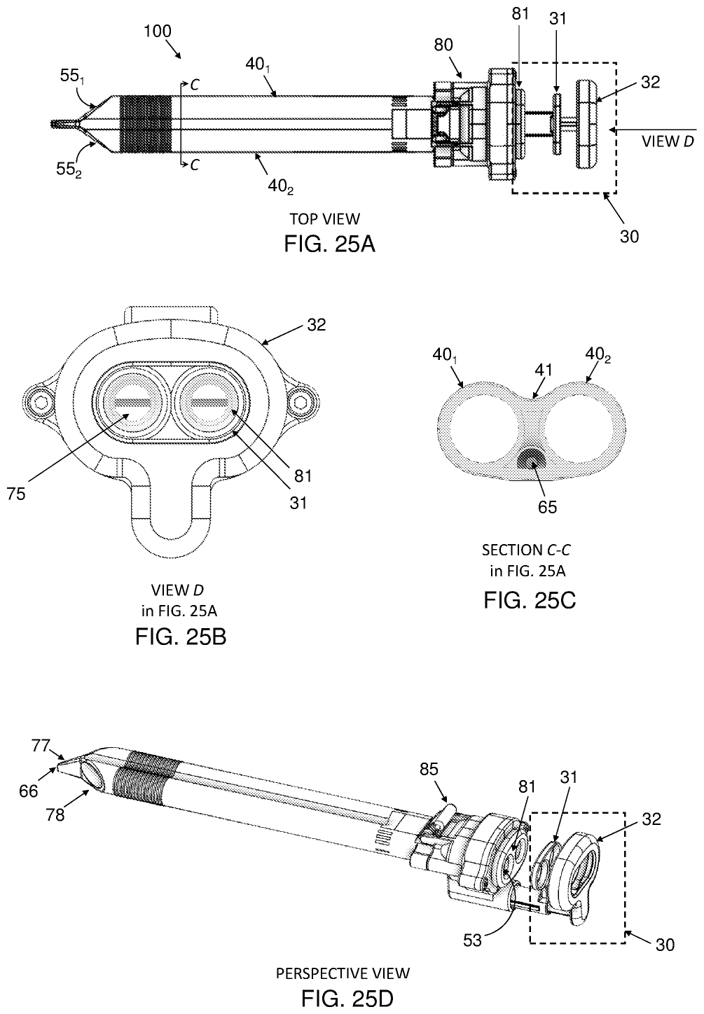

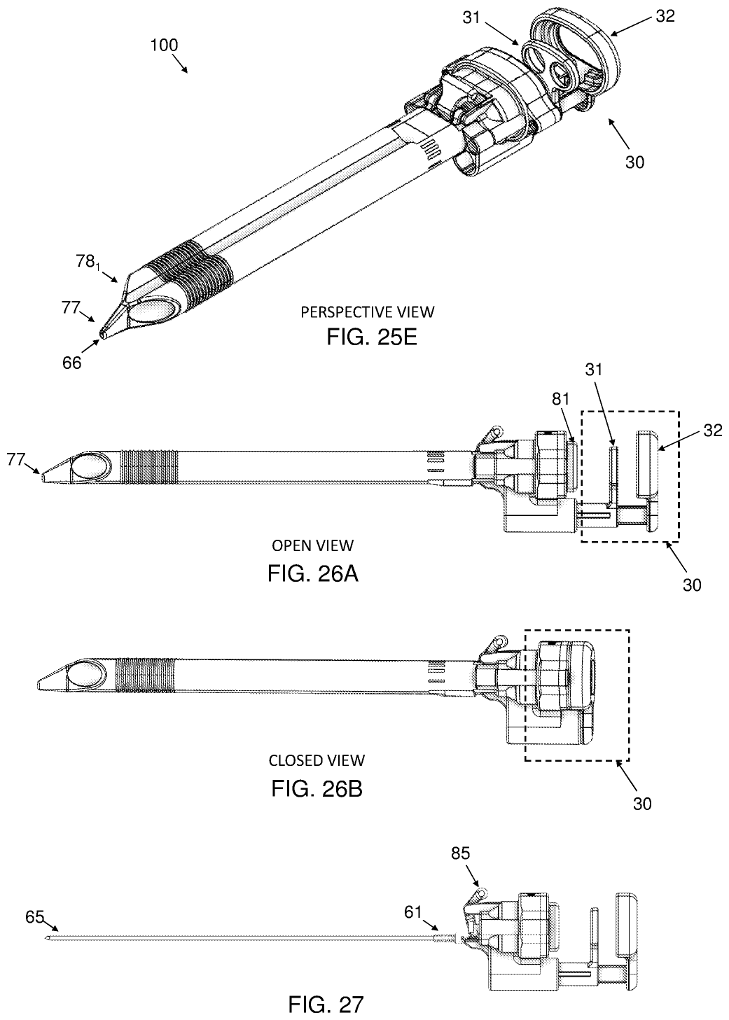

Reference is made to FIGS. 25A-E, 26A-B, 27 and 28A-B, in which various schematic illustrations of an access device 100 according to embodiments disclose features and configurations which can be implemented in combination with any one or more of the features of any of the access devices 100 disclosed herein.

FIGS. 25A-E show various views of an exemplary access device 100 comprising a sighting assembly 30. The sighting assembly 30 includes one or more elements which are effective to aid aligning the surgical arms 110 of a surgical robot 150 with the conduits 40 of the access device 100. In the instant non-limiting example, first and second sighting elements 32, 31 are sliding elements provided for aligning surgical arms 110 with the conduits 40. As shown in FIG. 25B, the sighting elements 32, 31 can be arranged with decreasing inner diameters, i.e., the inner diameters of the second element 31 are smaller than the inner diameters of the first element 32. The inner diameters of proximal entry seals 81 of the proximal base member 80 are, in turn, smaller than the inner diameters of the second sighting element 31. In some embodiments, the proximal entry seals 81 of the proximal base member 80 are configured, in terms of inner diameter and/or material selection, to seal around surgical arms 110 when one or more of said arms are present, or to seal around an intermediate element holding or surrounding the arms. Thus, the space provided within the second sighting element 31 is larger in diameter than the surgical arms 110. In embodiments, the sighting elements 32, 31 are provided for aligning with the conduits 40 of the access device 100 (or any other passageway for the arms used in accessing the surgical sight), and the arms are not expected to lean on the sighting elements 32, 31 or come in contact with them. The shapes shown in FIG. 25B for the first and second sighting elements 32, 31 are merely illustrative, and in some designs can have other shapes, such as, for example, open shapes that are subsets of the illustrated shapes, e.g., partial or open loops or ovals. In such cases, the ‘inner diameter’ refers to that or any arc or expanse described by the partial or open loops or ovals.

FIG. 25C shows a cross-section of the conduits 40 of the access device 100, featuring a puncturing needle 65 offset from the axis of the conduits 40₁, 40₂, as an alternative to the exemplary design of FIG. 7B where the puncturing needle 65 is centered between the cannulas (conduits 40₁, 40₂). As can be seen in FIGS. 25D and 25E, the aperture 66 at the distal tip 77 of the access device 100 for passing the puncturing needle 65 therethrough is accordingly lowered in comparison to the exemplary design of FIGS. 14A and 15A. Inter alia, the offset needle design allows the conduits to be placed closer together than if the needle 65 were between the conduits 40. FIG. 25D also shows an actuation member 85 for the puncturing needle 65, disposed distally from the base unit 80 and not at its proximal end as in the example of FIGS. 16B and 16D. Exemplary linkage between the actuation member 85 and the puncturing needle 65 is shown in the partial view of FIG. 27. In another design option (not shown) the actuation member 85 can be accessible on the bottom surface of the access device 100 rather than on the top surface as in FIG. 25D. The actuation member 85 can also be fitted with a safety mechanism, such as a latch (not shown) to prevent inadvertent actuation of the puncturing needle 65.

In some embodiments, the sighting assembly 30 can be telescopic, e.g., one-stage, two-stage, or three-stage telescopic; a two-stage telescopic sighting assembly 30 is illustrated in FIGS. 26A-B. The sighting assembly 30 is shown in an extended state in FIG. 26A, and in a compacted state in FIG. 26B. The total sliding extension of the sighting assembly 30 can be at least 30 mm, or at least 40 mm, or at least 50 mm, or at least 60 mm, or at least 70 mm, or at least 80 mm. In an exemplary use case the sighting assembly 30 is in the extended state while lining up the surgical arms 110, such that first and second elements 32, 31 are at their maximum extension, and the sighting assembly 30 is in the compacted state while carrying out a surgical procedure, e.g., to achieve maximum extension of the surgical arms 110 towards a surgical site.

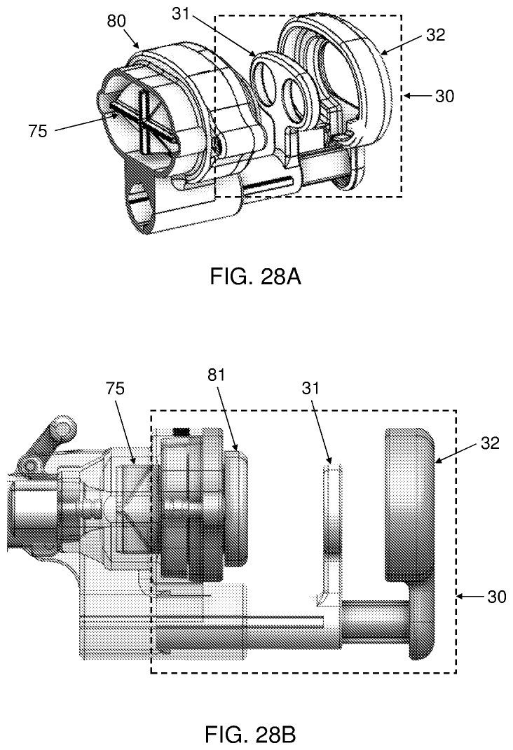

FIGS. 28A-B illustrate an optional configuration in which a single one-way valve 75, e.g., a duckbill valve, accommodates the passage of two surgical arms 110, as opposed to the two-valve design shown, e.g., in FIGS. 8B and 16B.

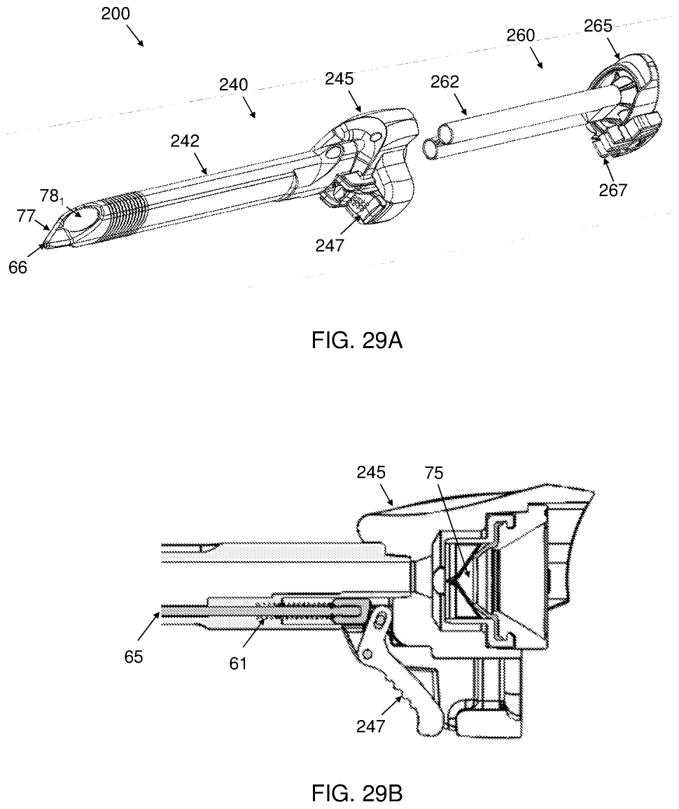

Attention is now drawn to FIGS. 29A to 31B, in which yet another embodiment of the access device in the form of apparatus 200, is shown in various positions and comprising a distal cannula member 240 that includes an activation member 247, and a proximal base member 260 that includes a tool-entry section 265 comprising a blocking element 267. The proximal base member 260 also includes a plurality of conduits 262 shaped for passage therethrough of surgical arms and tools. The plurality of conduits 262 are shaped to fit inside a sheath section 242 of the distal cannula member 240. In FIGS. 29A-29B, the proximal base member 260 is not yet loaded into the distal cannula member 240, giving the actuation member 247 full range of motion to transition between a depressed and an undepressed state.

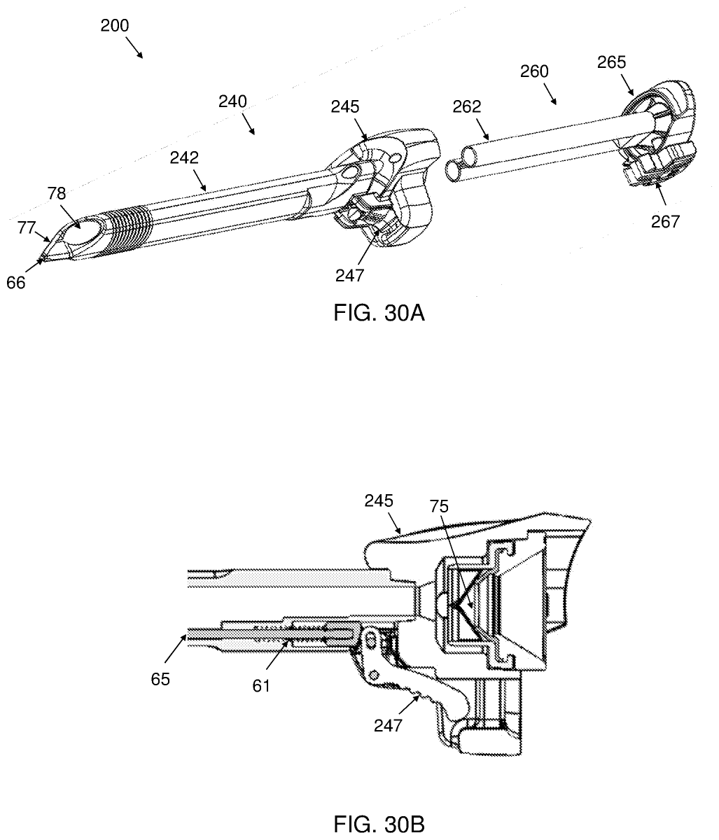

With particular reference to FIGS. 30A-30B, when the proximal base member 260 is not yet loaded into the distal cannula member 240, the actuation member can be depressed in order to activate the puncturing needle 65 (shown FIG. 30B). The actuation member 247 is depressed and the needle 65 is pushed forward so as to protrude from the aperture 66 in the distal tip 77 of the distal cannula member 240 in order to puncture the tissue, e.g., the tissue 97 of FIG. 20B.

Following the puncturing step, the actuation member 247 is released (back to its undepressed state), the needle is retracted back into a withdrawn position in the distal cannula member 240 and the procedure requires dilation of the puncture in order to introduce the distal cannula member 240 into the incision. Once the distal cannula member 240 has been inserted into the dilated incision, the proximal base member 260 may be introduced into the distal cannula member 240 to be joined therewith, e.g., by being snapped together. From this point onwards, the needle 65 is no longer required and, furthermore, may hinder the procedure if accidentally activated.

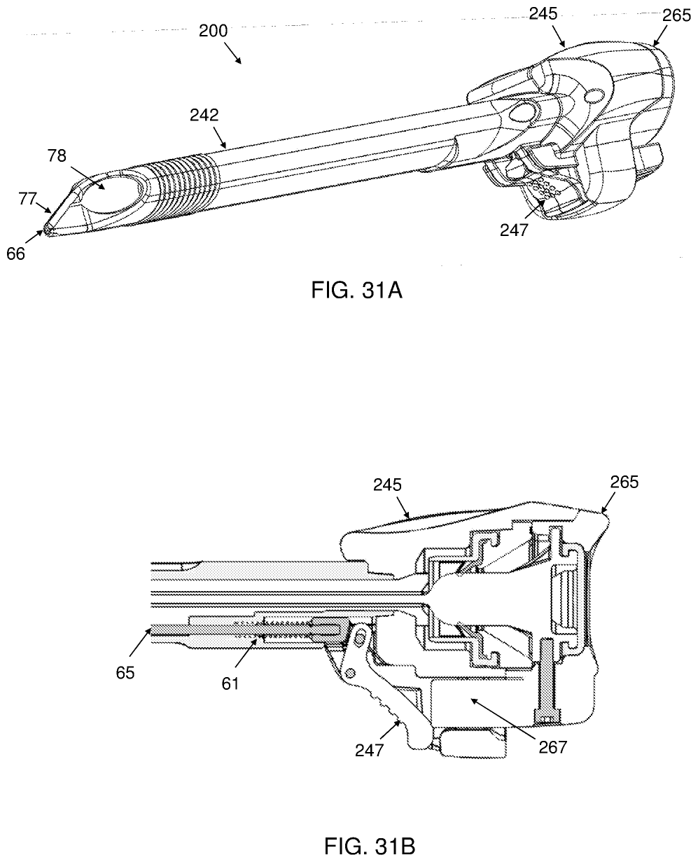

Turning now to FIGS. 31A-31B, once the proximal base member 260 is joined with the cannula 240, a blocking element 267 on the proximal base member 260 extends longitudinally such that it is positioned between the actuation member 247 and the body of the distal cannula member 240, thereby preventing depression of the actuation member 247. Consequently, as long as the proximal base member 260 is joined to, e.g., snapped together, the possibility of using the actuation member 247 is disabled, thereby preventing spontaneous protrusion of the needle 65 from the sheath and possible injury.

It is also noted that when the proximal base member 260 is introduced into the distal cannula member 240, the blocking element 267 also serves as a snap-fitting portion, allowing to maintain the proximal base member 260 the distal cannula member 240 joined together. Thus, the blocking element 267 carries a dual function: a snap fitting mechanism and, at the same time, a blocker preventing activation of the actuation mechanism of the puncturing needle.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A device for accessing a body cavity through a natural orifice, the device comprising a proximal base member and a plurality of conduits arranged to receive a corresponding plurality of surgical tools, and further comprising, for each conduit:
i. a lumen shaped for passage therethrough of a surgical tool, the lumen having a proximal port and a distal opening,
ii. a gas port in fluid communication with the lumen and displaced distally from the proximal port, and
iii. a proximal sealing gasket seated in the lumen and adapted for selectively opening and closing, the gasket being displaced distally from the proximal port and proximally from the gas port so as to create a respective proximally-sealed distal gas volume in communication with the body cavity.

2. The device of claim 1, wherein at least one of the gas ports is configured for insufflating therethrough a gas, from an external source of gas, into the body cavity, and at least one of the gas ports is configured for expelling insufflated gas therethrough, the respective proximally-sealed distal gas volumes of the plurality of conduits combining to form, in combination with the body cavity, a closed gas volume.

3. The device of either one of claim 1 or 2, wherein when a surgical tool is inserted into the body cavity through a respective conduit of the plurality of conduits, the distal gas volumes of both the respective conduit and at least one other conduit of the plurality of conduits remain proximally-sealed and in communication with the body cavity.

4. The device of claim 1, wherein when the plurality of surgical tools have passed through the respective lumens to access the body cavity, and a surgical tool of the plurality of surgical tools is withdrawn proximally from the body cavity and passes out of the respective lumen through the proximal port, the distal gas volumes of both the respective conduit and at least one other conduit of the plurality of conduits remain proximally-sealed and in communication with the body cavity.

5. The device of claim 2, wherein the closed gas volume (i) includes an insufflated gas and (ii) remains closed when a surgical tool is inserted into the body cavity through a respective conduit of the plurality of conduits.

6. The device of either one of claim 2 or 5, wherein the closed gas volume (i) includes an insufflated gas and (ii) remains closed when a surgical tool is removed from the body cavity and passes out of a respective lumen through the proximal port.

7. The device of any one of claims 1 to 3, wherein each of the lumens is entirely surrounded by a corresponding conduit.

8. The device of any one of claims 1 to 3, wherein a respective longitudinal portion of one or more of the lumens is surrounded by the proximal base member.

9. The device of any one of claims 1 to 3, wherein a respective longitudinal portion of one or more conduits of the plurality of conduits is surrounded by the proximal base member.

10. An assembly, comprising: (i) the device of any one of claims 1 to 9, (ii) for each respective gas port, a fluid conveyance in communication therewith and comprising a respective fluid valve, and (iii) a source of a gas for insufflation of the body cavity, arranged for insufflating the gas into the body cavity through at least one of the fluid conveyances.

11. The assembly of claim 10, wherein at least one of the fluid conveyances is arranged for expelling insufflated gas therethrough.

12. The assembly of either one of claim 10 or 11, wherein the respective proximally-sealed distal gas volumes of the plurality of conduits combine to form, in combination with the body cavity, a closed gas volume that remains closed when a surgical tool is inserted into the body cavity through a respective conduit of the plurality of conduits.

13. The assembly of claim 12, wherein the closed gas volume remains closed when a surgical tool is removed from the body cavity and passes out of a respective lumen through the proximal port.

14. The device of any one of claims 1 to 13, additionally comprising, for each conduit: a dilator, and an actuatable puncturing needle biased to a withdrawn position and arranged to pass through an aperture at a distal tip of the dilator.

15. The device of claim 14, wherein the biasing is by a biasing element disposed within the conduit.

16. The device of either one of claim 14 or 15, wherein the dilator is configured for passage through the proximal sealing gasket, such that when the dilator is withdrawn proximally from the body cavity and passes out of the respective conduit through the proximal port, the distal gas volume of at least one other conduit of the plurality of conduits remains proximally-sealed and in communication with the body cavity.

17. The device of any one of claims 14 to 16, wherein the closed gas volume remains closed when the dilator is removed from the body cavity and passes out of a respective lumen through the proximal port.

18. The device of any one of claims 14 to 17, wherein the puncturing needle is arranged to be withdrawn together with the dilator.

19. The device of any one of claims 14 to 18, additionally comprising an actuation member arranged to actuate, simultaneously, the respective puncturing needles of the plurality of conduits.

20. The device of claim 19, wherein the actuation member is configured for being withdrawn from the device together with the respective puncturing needles and the respective dilators of the plurality of conduits.

21. The device of any one of claims 14 to 20, wherein the conduits comprise respective tubes.

22. The device of any one of claims 1 to 13, wherein the conduits are distally beveled to form, in combination, a section of a dilator.

23. The device of any one of claim 1 to 13, or 22, additionally comprising an actuatable puncturing needle biased to a withdrawn position and arranged to pass through an aperture at a distal tip of the device.

24. The device of any one of claims 1 to 13, or 22 to 23, wherein the distal tip of the device is at least partly beveled to form a section of a dilator.

25. The device of any one of claims 1 to 13, or 22 to 24, wherein the biasing is by one or more biasing elements disposed outside the conduits.

26. The device of claim 25, additionally comprising an actuation member arranged to actuate the puncturing needle by overcoming a mechanical resistance of the one or more biasing elements.

27. The device of claim 26, additionally comprising an activation-prevention mechanism preventing actuation of the puncturing needle without an initial action that does not cause actuation.

28. The device of claim 27, wherein the actuation member is displaceable to expose the proximal ports for receiving the surgical tools.

29. The device of any one of claims 1 to 28, wherein the plurality of conduits comprises exactly two conduits.

30. A device for accessing a body cavity through a natural orifice, the device comprising a proximal base member and a plurality of conduits arranged to receive a corresponding plurality of surgical tools, and further comprising, for each conduit: (i) a lumen shaped for passage therethrough of a surgical tool, the lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity, the respective proximally-sealed distal gas volumes of the plurality of conduits combining to form, in combination with the body cavity, a closed gas volume, wherein the closed gas volume is effective to remain closed when any one or more of the following events occur: (i) a surgical tool is inserted into the body cavity through a respective conduit of the plurality of conduits, and (ii) a surgical tool is removed from the body cavity and passes out of a respective lumen through the proximal port.

31. The device of claim 30, wherein when the closed gas volume includes an insufflated gas, the closed gas volume is effective to remain closed when any one or more of the following events occur: (i) a surgical tool is inserted into the body cavity through a respective conduit of the plurality of conduits, and (ii) a surgical tool is removed from the body cavity and passes out of a respective lumen through the proximal port.

32. The device of either one of claim 30 or 31, wherein at least one of the gas ports is configured for insufflating therethrough a gas, from an external source of gas, into the closed gas volume, and at least one of the gas ports is configured for expelling insufflated gas therethrough.

33. The device of any one of claims 30 to 32, wherein when (i) the closed gas volume includes an insufflated gas, (ii) a surgical tool is removed from the body cavity and passes out of a respective lumen through the proximal port, and (iii) subsequent to the removal, a replacement surgical tool is inserted into the body cavity through the respective lumen, the closed gas volume is effective to retain substantially all of the insufflated gas.

34. A device for accessing a body cavity through a natural orifice, the device comprising:
a. a proximal base member;
b. a plurality of conduits arranged to receive a corresponding plurality of surgical tools for passage therethrough to the body cavity, the conduits distally beveled to form, in combination, a section of a dilator;
c. an actuatable puncturing needle biased to a withdrawn position by one or more biasing elements disposed outside the conduits and arranged to pass through an aperture at a distal tip of the device; and
d. an actuation member arranged to actuate the puncturing needle by overcoming a mechanical resistance of the one or more biasing elements.

35. The device of claim 34, wherein when the actuation member is released after an actuation, the puncturing needle is configured to return to the withdrawn position for storage at least during a use of the dilator to distally advance the device.

36. The device of claim 34, wherein when the actuation member is released after an actuation, the puncturing needle is configured to return to the withdrawn position for storage at least during passage of the surgical tools through the plurality of conduits to access the body cavity.

37. The device of any one of claims 34 to 36, additionally comprising an actuation-prevention mechanism preventing actuation of the puncturing needle without an initial action that does not cause actuation.

38. The device of any one of claims 34 to 37, wherein the actuation member is displaceable to expose the proximal ports for receiving the surgical tools.

39. The device of any one of claims 34 to 38, wherein the device comprises exactly one actuatable puncturing needle and exactly one actuation member.

40. The device of any one of claims 34 to 39, wherein the distal tip is formed integrally with the plurality of conduits.

41. The device of any one of claims 34 to 40, wherein the plurality of conduits are formed together and are not non-destructively separable.

42. The device of any one of claims 34 to 41, wherein the plurality of conduits comprises exactly two conduits.

43. The device of claim 42, wherein a transverse cross-section of the device intercepting the plurality of conduits at any point distal to the proximal base is such that a minimum-area circumscription of the cross-section is concave.

44. The device of any one of claims 34 to 43, further comprising, for each conduit: (i) a lumen shaped for passage therethrough of a surgical tool, the lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity, wherein at least one of the gas ports is configured for insufflating therethrough a gas, from an external source of gas, into the body cavity, and at least one of the gas ports is configured for expelling insufflated gas therethrough.

45. The device of claim 44, wherein (i) the respective proximally-sealed distal gas volumes of the plurality of conduits combine to form, in combination with the body cavity, a closed gas volume, and (ii) the closed gas volume is effective to remain closed when a surgical tool is inserted into the body cavity through a respective conduit of the plurality of conduits.

46. The device of claim 44, wherein the respective proximally-sealed distal gas volumes of the plurality of conduits combine to form, in combination with the body cavity, a closed gas volume, wherein the closed gas volume is oeprative to remain closed when a surgical tool is removed from the body cavity and passes out of a respective lumen.

47. The device of any one of claims 44 to 46, wherein the closed gas volume (i) includes an insufflated gas and (ii) remains closed when a surgical tool is inserted into the body cavity through a respective conduit of the plurality of conduits.

48. The device of any one of claims 44 to 47, wherein the closed gas volume (i) includes an insufflated gas and (ii) remains closed when a surgical tool is removed from the body cavity and passes out of a respective lumen through the proximal port.

49. A method of accessing a surgical site in a body cavity with multiple surgical tools through a natural orifice, the method comprising:
a. providing an access device comprising a proximal base member and a plurality of conduits, and further comprising, for each conduit: (i) a lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity;
b. inserting a distal portion of the access device through the orifice; and
c. introducing the multiple surgical tools into the body cavity through respective lumens of the access device.

50. The method of claim 49, wherein the proximally-sealed distal gas volumes remain proximally-sealed and in communication with the body cavity during the introducing.

51. The method of either one of claim 49 or 50, wherein the introducing includes introducing one surgical tool at a time.

52. The method of either one of claim 49 or 50, wherein the introducing includes introducing one or more of the multiple surgical tools in parallel.

53. The method of any one of claims 49 to 52, additionally comprising, after the introducing of the multiple surgical tools, proximally withdrawing a surgical tool of the multiple surgical tools from the body cavity and out of the respective lumen via the proximal port, the proximally-sealed distal gas volumes remaining proximally-sealed and in communication with the body cavity.

54. The method of any one of claims 49 to 53, wherein the surgical tools comprise robotic tools, and the introducing of the surgical tools includes controlling the movements of the robotic tools.

55. The method of any one of claims 49 to 54, additionally comprising: (i) placing an external gas source in communication with a respective gas port of the plurality of conduits via a fluid conveyance comprising a fluid valve, and (ii) insufflating a gas from the gas source into the body cavity.

56. The method of claim 55, additionally comprising: expelling insufflated gas through a gas port of the plurality of conduits into a fluid conveyance comprising a fluid valve.

57. The method of either one of claim 55 or 56, additionally comprising: replacing a surgical tool when the closed gas volume includes an insufflated gas, the closed gas volume being effective to remain closed when any one or more of the following events occur: (i) a surgical tool is introduced into the body cavity through a respective conduit of the plurality of conduits, and (ii) a surgical tool is removed from the body cavity and passes out of a respective lumen through the proximal port.

58. The method of any one of claims 49 to 57, wherein the plurality of conduits comprises exactly two conduits.

59. The method of any one of claims 49 to 58, wherein the provided access device includes one or more previously used conduits.

60. The method of any one of claims 49 to 59, wherein the access device additionally comprises, for each conduit: a dilator, and an actuatable puncturing needle biased to a withdrawn position and arranged to pass through an aperture at a distal tip of the dilator, the biasing being by a biasing element disposed within the conduit.

61. The method of claim 60, additionally comprising:
i. actuating, simultaneously, the respective puncturing needles of the plurality of conduits, using an actuation member of the access device,
ii. forming an incision in tissue which separates the body cavity from the orifice, and
iii. dilating the incision by advancing the distal portion of the access device therethrough to place respective distal openings of the conduits in communication with the body cavity.

62. The method of either one of claim 60 or 61, additionally comprising: proximally withdrawing the respective dilators of the plurality of conduits from the body cavity, and causing the respective dilators to pass out of the conduits, the proximally-sealed distal gas volumes remaining proximally-sealed and in communication with the body cavity.

63. The method of claim 62, wherein the puncturing needles and the biasing elements are withdrawn together with the dilators.

64. The method of any one of claims 49 to 63, wherein the conduits comprise respective tubes.

65. The method of any one of claims 49 to 59, wherein the conduits are distally beveled to form, in combination, a section of a dilator.

66. The method of any one of claim 49 to 59, or 65, wherein the access device additionally comprises an actuatable puncturing needle that is biased, by one or more biasing elements disposed outside the conduits, to a withdrawn position and arranged to pass through an aperture at a distal tip of the device, the distal tip of the device being at least partly beveled to form a section of a dilator.

67. The method of claim 66, additionally comprising:
i. actuating the puncturing needle using an actuation member arranged to overcome a mechanical resistance of the one or more biasing elements,
ii. forming an incision in tissue which separates the body cavity from the orifice, and
iii. dilating the incision by advancing the distal portion of the access device therethrough to place respective distal openings of the conduits in communication with the body cavity.

68. The method of either one of claim 66 or 67, wherein the access device additionally comprises an activation-prevention mechanism preventing actuation of the puncturing needle without an initial action that does not cause actuation.

69. The method of either one of claim 67 or 68, wherein the actuation member is displaceable to expose the proximal ports for receiving the surgical tools.

70. A method of accessing a surgical site in a body cavity with multiple surgical tools through a natural orifice, the method comprising:
a. providing an access device comprising: (i) a proximal base member, (ii) a plurality of conduits distally beveled to form, in combination, a section of a dilator, and (iii) a single actuatable puncturing needle disposed outside the conduits and biased to a withdrawn position by one or more biasing elements;
b. inserting, through the orifice, a distal portion of the access device;
c. actuating the puncturing needle using an actuation member arranged to overcome a mechanical resistance of the one or more biasing elements, wherein the actuating includes (i) passing a distal tip of the puncturing needle through an aperture at a distal tip of the device, and (ii) forming an incision in tissue which separates the body cavity from the orifice;
d. dilating the incision by distally advancing the access device therethrough to place respective distal openings of the conduits in communication with the body cavity; and
e. introducing the multiple surgical tools into the body cavity through respective conduits.

71. The method of claim 70, wherein when the actuation member is released after an actuation, the puncturing needle is configured to return to the withdrawn position for storage at least during the distally advancing.

72. The method of either one of claim 70 or 71, wherein when the actuation member is released after an actuation, the puncturing needle is configured to return to the withdrawn position for storage at least during the introducing.

73. The method of any one of claims 70 to 72, wherein the access device includes an actuation-prevention mechanism preventing actuation of the puncturing needle without an initial action that does not cause actuation, and actuating includes performing the initial action that does not cause actuation.

74. The method of any one of claims 70 to 73, additionally comprising: displacing the actuation member to expose the proximal ports for receiving the surgical tools.

75. The method of any one of claims 70 to 74, wherein the access device comprises exactly one actuatable puncturing needle and exactly one actuation member.

76. The method of any one of claims 70 to 75, wherein the plurality of conduits comprises exactly two conduits.

77. The method of any one of claims 70 to 76, wherein the distal tip of the access device is formed integrally with the plurality of conduits.

78. The method of any one of claims 70 to 77, wherein the plurality of conduits are formed together and are not non-destructively separable.

79. The method of any one of claims 70 to 78, wherein a transverse cross-section of the access device intercepting the plurality of conduits at any point distal to the proximal base is such that a minimum-area circumscription of the cross-section is concave.

80. The method of any one of claims 70 to 79, wherein
i. the access device further comprises, for each conduit: (i) a lumen shaped for passage therethrough of a surgical tool, the lumen having a proximal port and a distal opening, (ii) a gas port in fluid communication with the lumen and displaced distally from the proximal port, and (iii) a proximal sealing gasket seated in the lumen and adapted for selectively opening and closing, the gasket being displaced distally from the proximal port and proximally from the gas port so as to create a respective proximally-sealed distal gas volume in communication with the body cavity, and
ii. the method additionally comprises: insufflating a gas from an external source of gas through at least one of the gas ports and into the body cavity.

81. The method of claim 80, additionally comprising: expelling insufflated gas through at least one of the gas ports.

82. The method of either one of claim 80 or 81, wherein (i) the respective proximally-sealed distal gas volumes of the plurality of conduits combine to form, in combination with the body cavity, a closed gas volume, and (ii) the closed gas volume remains closed during the introducing.

83. The method of any one of claims 80 to 82, wherein (i) the respective proximally-sealed distal gas volumes of the plurality of conduits combine to form, in combination with the body cavity, a closed gas volume, (ii) the method additionally comprises: withdrawing a surgical tool, the withdrawing including removing the surgical tool from the body cavity and causing the surgical tool to pass out of a respective lumen, and (iii) the closed gas volume is effective to remain closed during the withdrawing.

84. The method of either one of claim 82 or 83, wherein the closed gas volume includes an insufflated gas.

85. An apparatus for accessing a body cavity through a natural orifice, the apparatus comprising:
a. a distal cannula assembly distally beveled to form a section of a dilator;
b. a proximal base member comprising a plurality of conduits shaped to be seated within the cannula member and receive therein a corresponding plurality of surgical tools for passage therethrough to the body cavity; and
c. an actuatable puncturing needle biased to a withdrawn position by a biasing element and arranged to pass through an aperture at a distal tip of the distal cannula assembly,
the distal cannula assembly comprising an actuation member arranged to actuate the puncturing needle by overcoming a mechanical resistance of the one or more biasing elements, the proximal base member comprising a blocking element shaped to block an actuating movement of the actuation member when the distal cannula assembly and proximal base member are joined.

86. The apparatus of claim 85, wherein the distal cannula assembly and proximal base member are shaped to snap together when joined.

87. The apparatus of either one of claim 85 or 86, wherein when the actuation member is released after an actuation, the puncturing needle is configured to return to a withdrawn position by the biasing element, and further to be restrained in said withdrawn position by the blocking element while the distal cannula assembly and proximal base member are joined.

88. A method of accessing a surgical site in a body cavity with multiple surgical tools through a natural orifice, the method comprising:
a. providing the apparatus of either one of claim 85 or 86;
b. inserting, through the orifice, a distal portion of the distal cannula member;
c. actuating the puncturing needle using the actuation member, wherein the actuating includes (i) passing a distal tip of the puncturing needle through the aperture at a distal tip of the device, (ii) forming an incision in tissue which separates the body cavity from the orifice, and (iii) releasing the actuation member such that the puncturing needle is returned by the biasing element to the withdrawn position;
d. joining the proximal base member and the distal cannula member, thereby blocking an additional actuating movement of the actuation member;
e. dilating the incision by distally advancing the apparatus therethrough to place respective distal openings of the conduits in communication with the body cavity; and
f. introducing the multiple surgical tools into the body cavity through respective conduits.

89. The method of claim 88, wherein the joining includes causing the proximal base member and the distal cannula member to snap together.
